(19) United States
(12) Patent Application Publication
(10) Pub. No.: US 2017/0202485 A1
(43) Pub. Date: Jul. 20, 2017

(54) PORTABLE ELECTRONIC APPARATUS AND DISPLAY METHOD FOR PORTABLE ELECTRONIC APPARATUS

(71) Applicant: SEIKO EPSON CORPORATION, SHINJUKU-KU (JP)

(72) Inventor: Kazuhiro TAKASU, Shiojiri-shi (JP)

(21) Appl. No.: 15/408,312

(22) Filed: Jan. 17, 2017

(30) Foreign Application Priority Data
Jan. 18, 2016 (JP) 2016-007283

(51) Int. Cl. A61B 5/11; G06F 3/02; A61B 5/024; A63B 71/06; A61B 5/083; A61B 5/0205; A63B 24/00; G06F 3/01; A61B 5/00

(52) U.S. Cl. CPC A61B 5/1118; G06F 3/014; G06F 3/0202; A61B 5/02438; A61B 5/4266; A61B 5/4815; A61B 5/0833; A61B 5/742; A61B 5/681; A61B 5/0205; A63B 24/0062; A63B 24/0003; A63B 71/0619; A61B 2562/0219; A63B 2220/20; A63B 2220/40; A63B 2230/06; A63B 2071/0663

(57) ABSTRACT

A portable electronic apparatus includes an input section that receives an input of an operation from a user, a sensor that performs detection related to physical activity of the user, a display section that displays data by using an output from the sensor, and a processing section that maintains a display object item of data displayed on the display section to be a predetermined item and changes a display object period of the data displayed on the display section to a period (differing period) whose length is different from a length of a predetermined period in a case where the input section receives an input of a first operation when the data in the predetermined period regarding the predetermined item is displayed on the display section.

### CROSS-REFERENCE TO RELATED APPLICATIONS

The entire disclosure of Japanese Patent Application No. 2016-007283, filed Jan. 18, 2016 is expressly incorporated by reference herein.

### BACKGROUND

1. Technical Field

The present invention relates to a portable electronic apparatus and a display method for the portable electronic apparatus.

2. Related Art

JP-A-2011-8612 discloses an activity meter which displays various data regarding an amount of activity such as energy consumption or an accumulated number of steps of a subject (walker). In the activity meter disclosed in JP-A-2011-8612, data other than predetermined data which is set in advance is displayed, and then a display format for the predetermined data is automatically displayed if an operation is not performed for a predetermined period of time. Consequently, it is possible to save the time and effort for the subject to perform an operation.

However, this activity meter requires a waiting time in order to switch between display of data other than the predetermined data and display of the predetermined data, and, thus, for example, a user who wants to compare the data of two types may feel troublesome. An activity meter which is a portable apparatus is restricted in a screen size unlike an installation type apparatus such as a personal computer, and thus there is concern that it may be hard to view each piece of data in a case where display of the number of steps for a day and display of an accumulated number of steps for a plurality of days are performed together on the same screen.

### SUMMARY

An advantage of some aspects of the invention is to provide a portable electronic apparatus which is convenient for a user to use, a display method for the portable electronic apparatus, and a display program for the portable electronic apparatus.

In the present specification, “data related to physical activity” includes both data regarding an amount of activity in a user's life and data regarding performance in the user's motion.

The invention can be implemented as the following forms or application examples.

### APPLICATION EXAMPLE 1

A portable electronic apparatus according to this application example includes an input section that receives an input of an operation from a user; a sensor that performs detection related to physical activity of the user; a display section that displays data by using an output from the sensor; and a processing section that maintains a display object item of data displayed on the display section to be a predetermined item and changes a display object period of the data displayed on the display section to a period (differing period) whose length is different from a length of a predetermined period in a case where the input section receives an input of a first operation when the data in the predetermined period regarding the predetermined item is displayed on the display section.

It is noted that “maintaining a display object item to be a predetermined item” includes not only “maintaining a display object item to be the same item as the predetermined item” but also “maintaining a display object item to be an item of the same type as that of the predetermined item”, “maintaining a display object item to be an item of the same type as that of the predetermined item but omitting or adding a part of display”, “maintaining a display object item to be the predetermined item but changing a display aspect of data”, and the like.

The portable electronic apparatus has restriction in a size of a display section unlike an installation type apparatus such as a personal computer, and, thus, if a large amount of data is simultaneously displayed on the same screen, there is concern that it may be hard to view each piece of data. However, in a case where an input of the first operation is received when the data in the predetermined period regarding the predetermined item is displayed, the processing section maintains a display object item of the data to be the predetermined item and changes a display object period of the data to a period whose length is different from a length of the predetermined period. In this case, the user can switch between display object periods while maintaining a display object item of the data by performing the first operation. Therefore, the user can compare pieces of data in different periods with respect to the same item on the basis of a change of the display content before and after the first operation.

### APPLICATION EXAMPLE 2

In the portable electronic apparatus according to the application example, the first operation may be formed of a single action.

Since the first operation is formed of a single action, the user can compare pieces of data in different periods with each other through a simple operation.

### APPLICATION EXAMPLE 3

In the portable electronic apparatus according to the application example, the input section may include a button, and the first operation may be an operation of pressing the button once.

Since the first operation is an operation of pressing the button once, the user can compare pieces of data in different periods with each other through a simple operation of pressing the button once.

### APPLICATION EXAMPLE 4

In the portable electronic apparatus according to the application example, in a case where the input section receives an input of a second operation when data in the differing period regarding the predetermined item is displayed on the display section, the processing section may maintain the display object item to be the predetermined item, and change the display object period from the differing period to the predetermined period.

In a case where the input section receives an input of the second operation when data in the differing period regarding the predetermined item is displayed, the processing section maintains the display object item of the data to be the predetermined item, and changes the display object period of the data from the differing period to the predetermined period. Therefore, the user can return the display object period of the data from the differing period to the predetermined period by performing the second operation after the first operation.

### APPLICATION EXAMPLE 5

In the portable electronic apparatus according to the application example, the second operation may be formed of a single action.

Since the second operation is formed of a single action, the user can return the display object period of the data from the differing period to the predetermined period through a simple operation.

### APPLICATION EXAMPLE 6

In the portable electronic apparatus according to the application example, the input section may include a button, and the second operation may be an operation of pressing the button once.

Since the second operation is formed of a single action of pressing the button once, the user can return the display object period of the data from the differing period to the predetermined period through a simple operation of pressing the button once.

### APPLICATION EXAMPLE 7

In the portable electronic apparatus according to the application example, the second operation may be the same as the first operation.

Since the second operation is the same as the first operation, the user can easily perform an operation of changing a display object period of the data from a predetermined period to a differing period, and an operation of changing the display object period of the data from the differing period to the predetermined period.

### APPLICATION EXAMPLE 8

In the portable electronic apparatus according to the application example, the predetermined period may be a part of the differing period.

Since the predetermined period is a part of the differing period, the user can compare data in a relatively long period with data in a relatively short period, periods of the pieces of data overlapping each other.

### APPLICATION EXAMPLE 9

In the portable electronic apparatus according to the application example, the predetermined period may be a day, and the differing period may be a week.

Since the predetermined period is a day, and the differing period is a week, the user can compare data for a day with data for a week.

### APPLICATION EXAMPLE 10

In the portable electronic apparatus according to the application example, the display object item may include at least one of an item regarding an amount of activity in the life and an item regarding performance in motion.

Since the display object item includes at least one of an item regarding an amount of activity in the life and an item regarding performance in motion, the user can check at least one of activity amount data and performance data.

### APPLICATION EXAMPLE 11

In the portable electronic apparatus according to the application example, the item regarding an amount of activity may include at least one of a distance, a time, the number of steps, a pace, a pitch, a stride, the number of increasing stories, the number of increasing stairs, a heartbeat, oxygen intake, an amount of perspiration, water intake, calorie consumption, calorie intake, calories balance, a weight, a waist size, a sleeping time, sleep quality, balance between sleep quality, balance between an excitation time and a relaxing time, a target achievement level, and an ultraviolet quantity.

At least one of the items indicates activity in the user's life. Therefore, the user can refer to data regarding at least one of the items for the user's life.

### APPLICATION EXAMPLE 12

In the portable electronic apparatus according to the application example, the item regarding performance may include at least one of a distance, a time, the number of steps, a pace, a pitch, a stride, increasing altitude, decreasing altitude, an elevation, gradient, the number of times of training, a target achievement level, an attitude, left/right difference, foot contact time, a directly-below landing ratio, propulsion efficiency, slow turnover of the legs, a landing brake quantity, landing impact, a heart rate, oxygen intake, an amount of perspiration, water intake, calorie consumption, an expected motion distance under a predetermined condition, the time until reaching a predetermined heartbeat zone, an expected pace under a predetermined condition, an expected pitch under a predetermined condition, an expected stride under a predetermined condition, an expected time under a predetermined condition, expected calorie consumption under a predetermined condition, an automatically generated target, and an ultraviolet quantity.

At least one of the items indicates the user's motion. Therefore, the user can refer to data regarding at least one of the items for the user's motion.

### APPLICATION EXAMPLE 13

In the portable electronic apparatus according to the application example, the portable electronic apparatus may be attachable to a predetermined part of the user.

Therefore, the user can perform physical activity in a state of carrying the portable electronic apparatus even without holding the portable electronic apparatus with the hand.

### APPLICATION EXAMPLE 14

In the portable electronic apparatus according to the application example, the predetermined part is the arm or the wrist of the user.

Therefore, the user can use the portable electronic apparatus with the same feeling as in a wristwatch or the like.

### APPLICATION EXAMPLE 15

A display method for a portable electronic apparatus according to this application example includes causing an input section to receive an input of an operation from a user; causing a sensor to perform detection related to physical activity of the user; causing a display section to display data by using an output from the sensor; and maintaining a display object item of data displayed on the display section to be a predetermined item and changing a display object period of the data displayed on the display section to a period (differing period) whose length is different from a length of a predetermined period in a case where an input of a first operation is received when the data in the predetermined period regarding the predetermined item is displayed on the display section.

The portable electronic apparatus has restriction in a size of a display section unlike an installation type apparatus such as a personal computer, and, thus, if a large amount of data is simultaneously displayed on the same screen, there is concern that it may be hard to view each piece of data. However, in the display method, in a case where an input of the first operation is received when the data in the predetermined period regarding the predetermined item is displayed, a display object item of the data is maintained to be the predetermined item, and a display object period of the data is changed to a period whose length is different from a length of the predetermined period. In this case, the user can switch between display object periods while maintaining a display object item of the data by performing the first operation. Therefore, the user can compare pieces of data in different periods with respect to the same item on the basis of a change of the display content before and after the first operation.

### APPLICATION EXAMPLE 16

A display program for a portable electronic apparatus according to this application example causes a computer to execute: receiving an input of an operation from a user; performing detection related to physical activity of the user; displaying data by using an output from a sensor; and maintaining a display object item of displayed data to be a predetermined item and changing a display object period of the displayed data to a period whose length is different from a length of a predetermined period in a case where an input of a first operation is received when the data in the predetermined period regarding the predetermined item is displayed.

The portable electronic apparatus has restriction in a size of a display section unlike an installation type apparatus such as a personal computer, and, thus, if a large amount of data is simultaneously displayed on the same screen, there is concern that it may be hard to view each piece of data. However, in the display program, in a case where an input of the first operation is received when the data in the predetermined period regarding the predetermined item is displayed, a display object item of the data is maintained to be the predetermined item, and a display object period of the data is changed to a period whose length is different from a length of the predetermined period. In this case, the user can switch between display object periods while maintaining a display object item of the data by performing the first operation. Therefore, the user can compare pieces of data in different periods with respect to the same item on the basis of a change of the display content before and after the first operation.

### BRIEF DESCRIPTION OF THE DRAWINGS

The invention will be described with reference to the accompanying drawings, wherein like numbers reference like elements.

FIG. 1 is a diagram for explaining an outline of an electronic apparatus according to a first embodiment.

FIG. 2 is a functional block diagram for explaining a configuration of a system including the electronic apparatus.

FIG. 3 is a flowchart for explaining a process in an information terminal related to data transmission with the electronic apparatus.

FIG. 4 is a flowchart for explaining a process in the information terminal related to information communication with a server.

FIG. 5 illustrates an example of an achievement level screen.

FIG. 6 illustrates a motion time screen.

FIG. 7 illustrates an example of calorie consumption screen.

FIG. 8 illustrates an example of a number-of-steps screen.

FIG. 9 illustrates an example of a sleeping time screen.

FIG. 10 illustrates an example of a mental balance screen.

FIG. 11 is a diagram illustrating transition of a screen in an activity amount display mode.

FIG. 12 is a flowchart illustrating a process in a processing section of the electronic apparatus in the activity amount display mode.

### DESCRIPTION OF EXEMPLARY EMBODIMENTS

Hereinafter, preferred embodiments of the invention will be described with reference to the drawings. The embodiments described below are not intended to improperly limit the content of the invention disclosed in the appended claims. In addition, all constituent elements described below are not essential constituent elements of the invention.

### 1. Embodiment of Electronic Apparatus

### 1-1. Outline of Electronic Apparatus

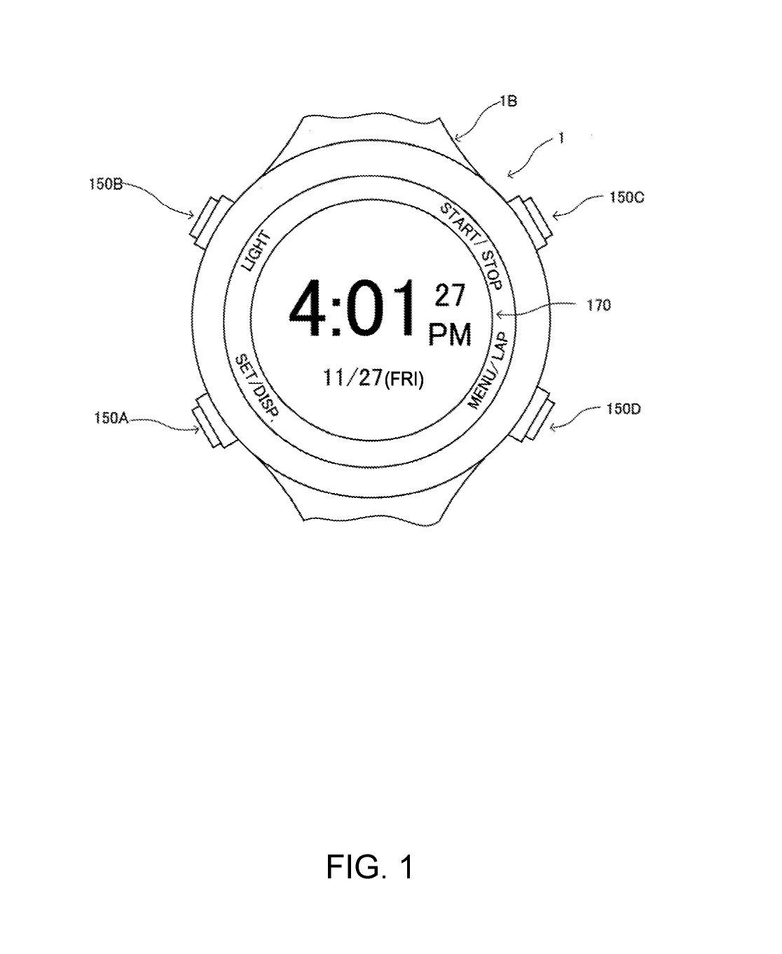

FIG. 1 is a diagram for explaining an outline of an electronic apparatus according to a first embodiment.

As illustrated in FIG. 1, an electronic apparatus 1 (an example of a mountable portable electronic apparatus) according to the present embodiment is a portable information apparatus mounted on a part of a user's body. A mounting location of the electronic apparatus 1 is, for example, a part (forearm) (an example of a predetermined part) between the elbow and the hand so as to be able to be viewed by the user.

In the example illustrated in FIG. 1, the electronic apparatus 1 is configured as a wrist type (wristwatch type) portable information apparatus, and has a belt 1B which is a mounting tool for mounting the electronic apparatus 1 on the user's wrist. For example, four operation buttons 150B, 150A, 150C and 150D formed of mechanical switches are provided at an outer edge of a display section 170 of the electronic apparatus 1. The operation buttons are an example of an input section which receives an input operation.

Here, if respective directions with the center of the display section 170 as a reference are expressed as time points assuming that the display section 170 is a dial of a 12 dual-hours type timepiece, in FIG. 1, a position of the operation button 150B in the display section 170 is a position of ten o'clock of the display section 170, a position of the operation button 150A is a position of eight o'clock of the display section 170, a position of the operation button 150C is a position of two o'clock of the display section 170, and a position of the operation button 150D is a position of four o'clock of the display section 170. A shape of the display section 170 is not limited to a disk shape illustrated in FIG. 1, and positions and the number of operation buttons 150B to 150D in the display section 170 are not limited to ones illustrated in FIG. 1.

Meanwhile, FIG. 1 illustrates a state in which the electronic apparatus 1 is in a time display mode. For example, the present date and time, a day of the week, AM or PM, and the like are displayed as numerical value images on the display section 170 in the time display mode.

A function for a user inputting an instruction for lighting or putting out a light source to the electronic apparatus 1 is assigned to the operation button 150B.

A function for the user inputting an instruction for switching between modes to the electronic apparatus 1 is assigned to the operation button 150A. However, when the electronic apparatus 1 is in a menu display mode (during a period in which a menu screen is displayed), a function for the user inputting a determination instruction (setting instruction) to the electronic apparatus 1 may be assigned to the operation button 150A.

A function for the user inputting a measurement starting instruction or a measurement finishing instruction of the split time or the like to the electronic apparatus 1 is assigned to the operation button 150C. When the electronic apparatus 1 is in the menu display mode (during a period in which a menu screen is displayed), a function for inputting an instruction for moving a cursor upward in the menu screen to the electronic apparatus 1 may be assigned to the operation button 150C.

A function for inputting an instruction for calling a menu screen to the electronic apparatus 1 is assigned to the operation button 150D. However, during measurement of the split time, a function for the user inputting an instruction for recording a lap time to the electronic apparatus 1 is assigned to the operation button 150D. When the electronic apparatus 1 is in the menu display mode (during a period in which a menu screen is displayed), a function for inputting an instruction for moving a cursor downward in the menu screen to the electronic apparatus 1 may be assigned to the operation button 150D.

The electronic apparatus 1 has not only a clocking function but also various sensing functions such as a pulse detection function, and thus operates as an activity meter. The electronic apparatus 1 as an activity meter measures an amount of activity (activity amount measurement) of the user by using the various sensing functions. Here, the “amount of activity” includes items regarding biological information of the user and items regarding behavior information of the user. The term “pulse” indicates pulsation occurring when a change in beating (heartbeat) at which the heat sends blood to the whole body is sent to the respective parts of the body. In the following description, the term “pulse” may be replaced with a “heartbeat” as appropriate within the scope of being technically replaceable.

The items regarding biological information include, for example, a sleeping time and a mental balance (the “mental balance” indicates a balance between a state of the user being excited and a state of the user relaxing, and is, for example, a ratio between time in a state in which the user is excited during non-motion and time in a state in which the user relaxes during non-motion). A pulse sensor, an acceleration sensor, an angular velocity sensor, and the like are used to measure the items. The sensors may be used to measure the biological information in combination with other sensors, and some of the sensors may not be used.

The items regarding behavior information include, for example, the number of steps, a motion time, calorie consumption, a movement distance, and an achievement level (which will be described later). A pulse sensor, an acceleration sensor, an angular velocity sensor, a global positioning system (GPS) sensor, a geomagnetic sensor, an atmospheric pressure sensor, and the like which will be described later are used to measure the items. The sensors may be used to measure the behavior information in combination with other sensors, and some of the sensors may not be used.

The electronic apparatus 1 performs logging of data regarding each item (activity amount data). Here, the “logging” indicates that, for example, data is recorded in activity amount measurement. The “logging of activity amount data” indicates that history of activity amount data is recorded (that is, data is recorded in a time series, or each data item is recorded in a form of being able to be specified). The activity amount data is an example of data related to physical activity.

### 1-2. Outline of User Action

In pre-preparation, the user displays a menu screen on the display section 170 of the electronic apparatus 1, and inputs data (user physical data) regarding the user's body, such as a height, a weight, age, sex, and a body fat percentage. The user causes the electronic apparatus 1 to start activity amount measurement, also inputs a target for each item, and returns the electronic apparatus 1 to a time display mode, on the menu screen.

Targets (user target data) for the respective items which can be input by the user include, for example, a target for the number of steps (target number of steps), a target for motion time (target motion time), a target for calorie consumption (target calorie consumption), a target for a sleeping time (target sleeping time), a target for a mental balance (target mental balance), and a target for a movement distance (target movement distance). The targets for the respective items which can be used by the user are classified into targets (long-term targets) which are to be achieved within a week, and targets (short-term targets) which are to be achieved every day. However, the user may not necessarily input all of the plurality of targets to the electronic apparatus 1. For example, the user may input some of the plurality of targets, and may omit input of all of the targets. In a case where input of some or all of the plurality of targets is omitted, display of some or all of the targets is omitted.

Then, the user lives, for example, for a week in a state of mounting the electronic apparatus 1 on the arm. The electronic apparatus 1 performs the activity amount measurement. During the activity amount measurement, the electronic apparatus 1 may or not display a clock screen. The user may display activity amount data on the display section 170 of the electronic apparatus 1 as appropriate within the user's daily life so as to obtain a chance to improve the life (details of display of the activity amount data will be described later).

For example, after a week from starting of the activity amount measurement, the user displays a menu screen on the display section 170 of the electronic apparatus 1, and returns the electronic apparatus 1 to the time display mode by finishing the activity amount measurement in the electronic apparatus 1.

The user may connect the electronic apparatus 1 to an information terminal such as a smart phone, a tablet PC, a desktop PC via short-range wireless communication (near field communication) or the like, so as to transmit log data (user activity amount data), user target data, and user physical data accumulated in the electronic apparatus 1 to the information terminal.

The user may connect the information terminal to a server via a network such as the Internet so as to upload the user activity amount data, the user target data, and the user physical data to the server and to be stored in the server.

The user may connect the information terminal to the server via a network such as the Internet at a desired timing, so as to check the user activity amount data thereof from the information terminal. At this time, the user may be provided with various additional information pieces (an application software program, map data, and the like) from the server.

For example, it is assumed that the user connects the information terminal to the server via a network such as the Internet in advance, so as to transmit registration information such as an ID (identification information) of the electronic apparatus 1 and an ID (identification information) of the user to the server, and thus registration of the user in the server is completed. After the registration, the user can be provided with a service of storing user activity amount data and the above-described additional information (the application software program, the map data, and the like) from the server.

### 1-3. Configuration of Electronic Apparatus and the Like

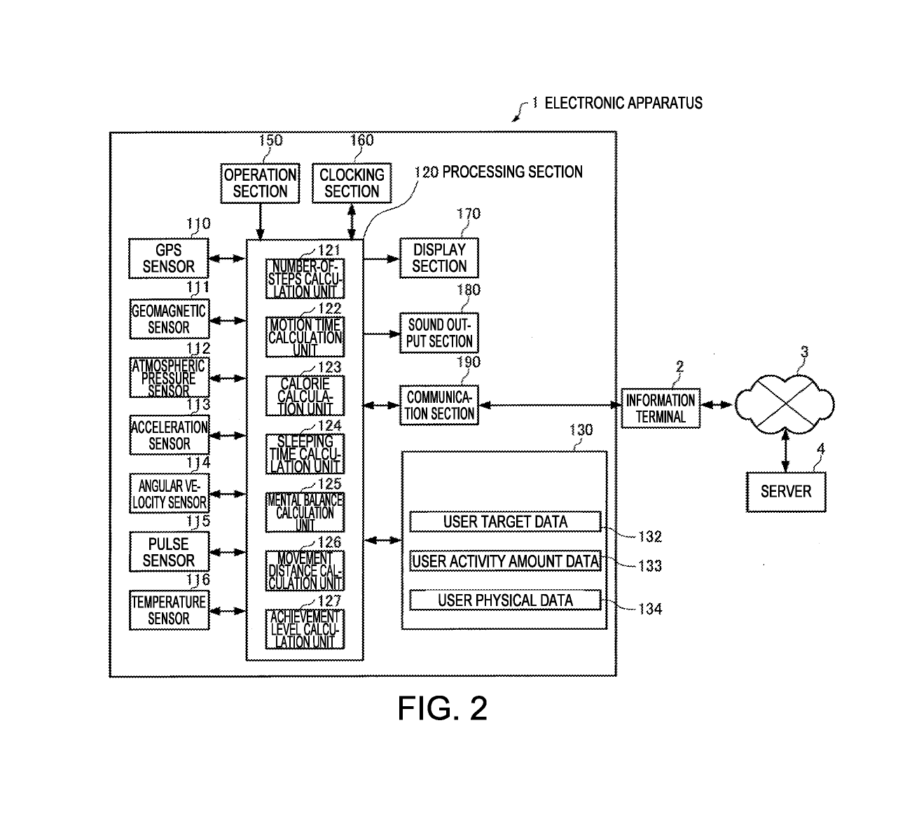

FIG. 2 is a functional block diagram for explaining a configuration of a system including the electronic apparatus.

The system of the present embodiment includes the electronic apparatus 1, an information terminal 2, and a server 4, and the information terminal 2 is connected to the server 4 via a network 3 such as the Internet. The electronic apparatus 1 can perform communication with the information terminal 2 via, for example, short-range wireless communication.

As illustrated in FIG. 2, the electronic apparatus 1 is configured to include a GPS sensor 110, a geomagnetic sensor 111, an atmospheric pressure sensor 112, an acceleration sensor 113, an angular velocity sensor 114, a pulse sensor 115, a temperature sensor 116, a processing section 120, a storage section 130, an operation section 150 (an example of an input section), a clocking section 160, a display section 170, a sound output section 180, a communication section 190, and the like. However, the electronic apparatus 1 may have a configuration in which some of the constituent elements are omitted or changed, or other constituent elements (for example, a humidity sensor and an ultraviolet sensor) are added thereto.

The GPS sensor 110 is a sensor which generates positioning data (data such as latitude, longitude, altitude, and a velocity vector) indicating a position and the like of the electronic apparatus 1 and outputs the data to the processing section 120 (examples of an input section and a processing section), and is formed of, for example, a global positioning system (GPS). The GPS sensor 110 receives an electromagnetic wave with a predetermined frequency band incoming from the outside by using a GPS antenna (not illustrated), extracts a GPS signal sent from a GPS satellite therefrom, and also generates positioning data indicating a position or the like of the electronic apparatus 1 on the basis of the GPS signal.

The geomagnetic sensor 111 is a sensor which detects a geomagnetic vector indicating a direction of a magnetic field of the earth, viewed from the electronic apparatus 1, and generates, for example, geomagnetic data indicating magnetic flux densities in three-axis directions which are orthogonal to each other. For example, a magnet resistive (MR) element, a magnet impedance (MI) element, or a hole element is used for the geomagnetic sensor 111.

The atmospheric pressure sensor 112 is a sensor which detects an ambient atmospheric pressure (atmospheric pressure), and includes, for example, a pressure sensitive element of a type (vibration type) using a change in a resonance frequency of a vibrator element. The pressure sensitive element is a piezoelectric vibrator made of a piezoelectric material such as quartz crystal, Lithium Niobate, or Lithium Tantalate, and employs, for example, a tuning fork type vibrator, a dual-tuning fork type vibrator, an AT vibrator (thickness shear vibrator), or a SAW vibrator. An output from the atmospheric pressure sensor 112 may be used to correct positioning data.

The acceleration sensor 113 is an inertial sensor which detects respective accelerations in the three-axis directions which intersect (are ideally orthogonal to) each other, and outputs digital signals (acceleration data) corresponding to magnitudes and directions of the detected three-axis accelerations. The outputs from the acceleration sensor 113 may be used to correct position information included in the positioning data from the GPS sensor 110.

The angular velocity sensor 114 is an inertial sensor which detects respective angular velocities in the three-axis directions which intersect (are ideally orthogonal to) each other, and outputs digital signals (angular velocity data) corresponding to magnitudes and directions of the detected three-axis angular velocities. The outputs from the angular velocity sensor 114 may be used to correct position information included in the positioning data from the GPS sensor 110.

The pulse sensor 115 is a sensor which generates a signal indicating a pulse of the user and outputs the signal to the processing section 120, and includes, for example, a light source such as a light emitting diode (LED) light source which applies measurement light having an appropriate wavelength toward a blood vessel under the skin, and a light receiving element which detects a change in the intensity of light generated at the blood vessel according to the measurement light. A light intensity change waveform (pulse wave) is processed according to a well-known method such as frequency analysis, and thus a pulse rate (a pulse rate per minute) can be measured. As the pulse sensor 115, instead of the photoelectric sensor formed of the light source and the light receiving element, an ultrasonic sensor which measures a pulse rate by detecting contraction of a blood vessel by using an ultrasonic wave, or a sensor which measures a pulse rate by causing a weak current from an electrode to flow into the body, may be employed.

The temperature sensor 116 is a temperature sensing element which outputs a signal (for example, a voltage corresponding to a temperature) corresponding to an ambient temperature. The temperature sensor 116 may output a digital signal corresponding to a temperature.

The processing section 120 is formed of, for example, a micro processing unit (MPU), a digital signal processor (DSP), and an application specific integrated circuit (ASIC). The processing section 120 performs various processes according to a program (an example of a display program) stored in the storage section 130, and various commands which are input by the user via the operation section 150. The processes in the processing section 120 include data processing on data which is generated by the GPS sensor 110, the geomagnetic sensor 111, the atmospheric pressure sensor 112, the acceleration sensor 113, the angular velocity sensor 114, the pulse sensor 115, the temperature sensor 116, the clocking section 160, and the like, display processing for displaying an image on the display section 170, sound output processing for outputting sounds from the sound output section 180. The processing section 120 functions as a number-of-steps calculation unit 121, a motion time calculation unit 122, a calorie calculation unit 123, a sleeping time calculation unit 124, a mental balance calculation unit 125, a movement distance calculation unit 126, and an achievement level calculation unit 127. Details of the number-of-steps calculation unit 121, the motion time calculation unit 122, the calorie calculation unit 123, the sleeping time calculation unit 124, the mental balance calculation unit 125, the movement distance calculation unit 126, and the achievement level calculation unit 127 will be described later.

The storage section 130 is formed of one or a plurality of IC memories, and includes a ROM storing data such as a program, and a RAM serving as a work area of the processing section 120. The RAM also includes a nonvolatile RAM, and the nonvolatile RAM secures storage regions for user target data 132, user activity amount data 133, user physical data 134, and the like. The user activity amount data 133 is stored in the storage section 130, for example, in the form of a data table. The data table stores, for example, sensing data for each item and for each period. The processing section 120 may perform switching between display object items, switching between display object periods, or the like, which will be described later on the basis of the data table.

The operation section 150 is formed of, for example, a button, a key, a microphone, and a touch panel so as to have a voice recognition function (using the microphone (not illustrated)) and an action detection function (using the acceleration sensor 113 or the like), and performs a process of converting an instruction from the user into an appropriate signal which is then sent to the processing section 120. The above-described operation buttons 150B, 150A, 150C and 150D are included in the operation section 150.

The clocking section 160 is formed of, for example, a real time clock (RTC) IC or the like, and generates time data such as year, month, day, hour, minute, and second, and sends the data to the processing section 120.

The display section 170 is formed of, for example, a liquid crystal display (LCD), an organic electroluminescence (EL) display, an electrophoretic display (EPD), or a touch panel display, and displays various images in response to instructions from the processing section 120.

The sound output section 180 is formed of, for example, a speaker, a buzzer, or a vibrator, and generates various sounds (or vibration) in response to instructions from the processing section 120.

The communication section 190 performs a variety of control for establishing data communication between the electronic apparatus 1 and the information terminal 2 (a smart phone or the like). The communication section 190 is configured to include a transceiver based on a short-range wireless communication standard such as Bluetooth (registered trademark) (including Bluetooth Low Energy (BTLE)), Wi-Fi (registered trademark) (Wireless Fidelity), Zigbee (registered trademark), NFC (Near Field Communication), or ANT+ (registered trademark).

The information terminal 2 is, for example, a smart phone, a tablet PC, or a desktop PC which can be connected to the network 3 such as the Internet, and includes a communication section (not illustrated) corresponding to the communication section 190 of the electronic apparatus 1. A program or the like for controlling the electronic apparatus 1 is installed in a storage section (not illustrated) of the information terminal 2, and the information terminal 2 is operated according to this program when connected to at least the electronic apparatus 1. The program is downloaded from, for example, the server 4 via the network 3 such as the Internet.

The server 4 is connected to the network 3 such as the Internet. The server 4 has a function of managing data or the like uploaded from a user of the electronic apparatus 1, for each user. The server 4 has a function of providing a program or map data to the user of the electronic apparatus 1.

### 1-4. Details of the Processing Section

Hereinafter, a description will be made of the number-of-steps calculation unit 121, the motion time calculation unit 122, the calorie calculation unit 123, the sleeping time calculation unit 124, the mental balance calculation unit 125, the movement distance calculation unit 126, and the achievement level calculation unit 127 of the processing section 120 in this order. A combination of the sensors to be used, described below, is only an example, and a well-known combination which can calculate a numerical value or the like of each item may be employed.

### 1-4-1. Calculation of Number of Steps

The number-of-steps calculation unit 121 counts the number of steps of the user on the basis of, for example, an output from the acceleration sensor 113, an output from the angular velocity sensor 114, and the user physical data 134. At least one of the acceleration sensor 113 and the GPS sensor 110 may be used to count the number of steps. The number-of-steps calculation unit 121 may determine whether or not a heart rate is included in a fat combustion zone on the basis of an output from the pulse sensor 115, and may count the number of steps when the heart rate is included in the fat combustion zone. The fat combustion zone is set on the basis of the user physical data 134. The fat combustion zone is disclosed in JP-A-2013-22256. The fat combustion zone may be determined on the basis of both a heart rate and accelerations.

The number-of-steps calculation unit 121 calculates the number of steps for each date and the number of steps for a week including a day to which the present time belongs, and writes the number of steps into the user activity amount data 133.

### 1-4-2. Calculation of Motion Time

The motion time calculation unit 122 calculates a normal motion time of the user on the basis of, for example, an output from the acceleration sensor 113 and an output from the angular velocity sensor 114. This calculation may be performed by using an output from the GPS sensor 110. The motion time calculation unit 122 may determine whether or not a heart rate is included in a fat combustion zone on the basis of an output from the pulse sensor 115, and may calculate a motion time when the heart rate is included in the fat combustion zone.

The motion time calculation unit 122 calculates a motion time for each date and motion times for a week including a day to which the present time belongs, and writes the motion times into the user activity amount data 133.

### 1-4-3. Calculation of Calorie

The calorie calculation unit 123 calculates calorie consumption on the basis of, for example, the user physical data 134 and an output from the pulse sensor 115. The calorie calculation unit 123 sets a basal metabolic rate of the user on the basis of the age, the sex, and the like included in the user physical data 134, and calculates calorie consumption on the basis of the basal metabolic rate. Calculation of the calorie consumption is performed according to a method as disclosed in JP-A-2009-285498 in which calorie consumption is calculated by using information regarding a pulse rate, age, and sex, or a well-known method in which a basal metabolic rate is obtained by using information regarding a weight or a height.

The calorie calculation unit 123 may compute a total value of calorie intake on the basis of meal information of the user so as to calculate calorie balance. The meal information of the user may be manually input by the user via, for example, the operation section 150.

The calorie calculation unit 123 calculates calorie consumption for each date and calorie consumption for a week including a day to which the present time belongs, and writes calorie consumption into the user activity amount data 133.

### 1-4-4. Calculation of Sleeping Time

The sleeping time calculation unit 124 calculates a sleeping time of the user on the basis of, for example, an output from the acceleration sensor 113, an output from the angular velocity sensor 114, and an output from the pulse sensor 115. The sleeping time calculation unit 124 determines whether or not the user is sleeping on the basis of an output from the acceleration sensor 113 and an output from the angular velocity sensor 114. The sleeping time calculation unit 124 may determine whether the user is in a light sleeping state or a deep sleeping state on the basis of an output from the pulse sensor 115 during the sleeping, and may calculate a light sleeping time and a deep sleeping time.

The sleeping time calculation unit 124 calculates a sleeping time for each date and sleeping times for a week including a day to which the present time belongs, and writes the sleeping times into the user activity amount data 133.

### 1-4-5. Calculation of Mental Balance

The mental balance calculation unit 125 calculates, for example, a ratio (mental balance) between time (excitation time) in a state in which the user is excited during non-motion and time (relaxing time) in a state in which the user relaxes during non-motion on the basis of, for example, an output from the acceleration sensor 113, an output from the angular velocity sensor 114, an output from the GPS sensor 110, and an output from the pulse sensor 115. A calculated excitation time may be an excitation time during motion, and a calculated relaxing time may be a relaxing time during motion.

In a case where a change in an output from the acceleration sensor 113 is not included in a motion acceleration range (a range of a change in acceleration divided into motions), and a measured pulse rate which is measured by the pulse sensor 115 is included in a motion pulse rate range (a range of a change in acceleration divided into motions), the mental balance calculation unit 125 determines that the user is in an excitation state (sympathetic nerve active state) not due to motion.

In a case where a change in an output from the acceleration sensor 113 is not included in a motion acceleration range (a range of a change in acceleration divided into motions), and a measured pulse rate which is measured by the pulse sensor 115 is not included in a motion pulse rate range (a range of a change in acceleration divided into motions), the mental balance calculation unit 125 determines that the user is in a relaxing state (parasympathetic nerve active state).

The mental balance calculation unit 125 may calculate an index HF/LF indicating active states of a sympathetic nerve and a parasympathetic nerve on the basis of a pulse wave measured by the pulse sensor 115, so as to determine an excitation state or a relaxing state.

The mental balance calculation unit 125 calculates a mental balance for each date and mental balances for a week including a day to which the present time belongs, and writes the mental balances into the user activity amount data 133.

### 1-4-6. Calculation of Movement Distance

The movement distance calculation unit 126 calculates a movement distance of the user on the basis of, for example, an output from the GPS sensor 110, an output from the geomagnetic sensor 111, an output from the atmospheric pressure sensor 112, an output from the acceleration sensor 113, and an output from the angular velocity sensor 114.

The movement distance calculation unit 126 can calculate a movement distance on the basis of only an output from the GPS sensor 110, but may not receive a GPS signal depending on an environment in which the electronic apparatus 1 is present. Therefore, for example, a movement distance calculated on the basis of an output from the GPS sensor 110 is corrected as appropriate on the basis of at least one of an output from the geomagnetic sensor 111, an output from the atmospheric pressure sensor 112, an output from the acceleration sensor 113, and an output from the angular velocity sensor 114, or a movement distance in a period in which a GPS signal cannot be received is estimated.

The movement distance calculation unit 126 calculates a movement distance for each date and movement distances for a week including a day to which the present time belongs, and writes the movement distances into the user activity amount data 133.

### 1-4-7. Calculation of Achievement Level

The achievement level calculation unit 127 calculates at least one of a ratio (an achievement level of the number of steps) of a calculated number of steps to the target number of steps, a ratio (an achievement level of a motion time) of a calculated motion time to the target motion time, a ratio (an achievement level of calorie consumption) of calculated calorie consumption to the target calorie consumption, a ratio (an achievement level of a sleeping time) of a measured sleeping time to the target sleeping time, a ratio (an achievement level of a mental balance) of a measured mental balance to the target mental balance, and a ratio (an achievement level of a movement distance) of a measured movement distance to the target movement distance, on the basis of the calculated number of steps, a motion time (calculated motion time), calorie consumption (calculated calorie consumption), a sleeping time (calculated sleeping time), a mental balance (calculated mental balance), and a movement distance (calculated movement distance), and the target number of steps, the target motion time, the target calorie consumption, the target sleeping time, the target mental balance, and the target movement distance included in the user target data 132.

The achievement level calculation unit 127 calculates achievement levels for each date and achievement levels for a week including a day to which the present time belongs, and writes the achievement levels into the user activity amount data 133.

### 1-5. Process in Information Terminal

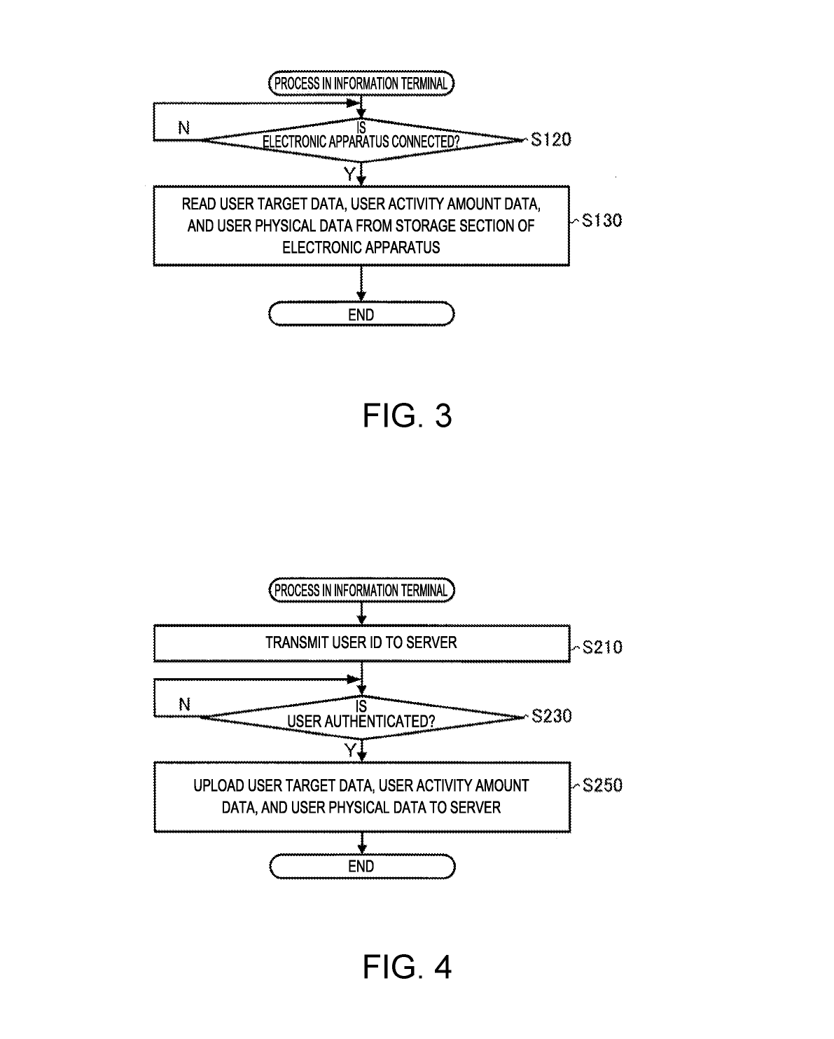

FIG. 3 is a flowchart for explaining a process in the information terminal 2 related to data transmission with the electronic apparatus 1. Hereinafter, each step in FIG. 3 will be described in order.

Step S120: The information terminal 2 determines whether or not communication can be performed with the electronic apparatus 1 via short-range wireless communication (that is, the information terminal 2 is connected to the electronic apparatus 1) (step S120), starts the next process (step S130) if it is determined that the information terminal 2 is connected to the electronic apparatus 1 (Y in step S120), and waits if otherwise (N in step S120).

Step S130: The information terminal 2 accesses the storage section 130 of the electronic apparatus 1 via the communication section 190 and the processing section 120 of the electronic apparatus 1, reads the user target data 132, the user activity amount data 133, and the user physical data 134 stored in the storage section 130, and finishes the flow (step S130). Transmission of the data (data transmission) from the electronic apparatus 1 to the information terminal 2 is performed in a predetermined format.

FIG. 4 is a flowchart for explaining a process in the information terminal 2 related to information communication with the server 4. Hereinafter, each step in FIG. 4 will be described in order.

Step S210: The information terminal 2 transmits a user ID to the server 4 via the network 3 (step S210).

Step S230: The information terminal 2 determines whether or not an access permission notification is received from the server 4 via the network 3 (step S230), starts the next process (step S250) if the access permission notification is received (if user authentication is completed) (Y in step S230), and waits if otherwise (N in step S230).

Step S250: The information terminal 2 transmits the user target data, the user activity amount data, and the user physical data to the server 4, and finishes the flow (step S250). Transmission of the data (information communication) from the information terminal 2 to the server 4 is performed in a predetermined format.

### 1-6. Process in Electronic Apparatus

### 1-6-1. Each Display Screen of Activity Amount Data

Hereinafter, a description will be made of display screens of activity amount data in the electronic apparatus 1.

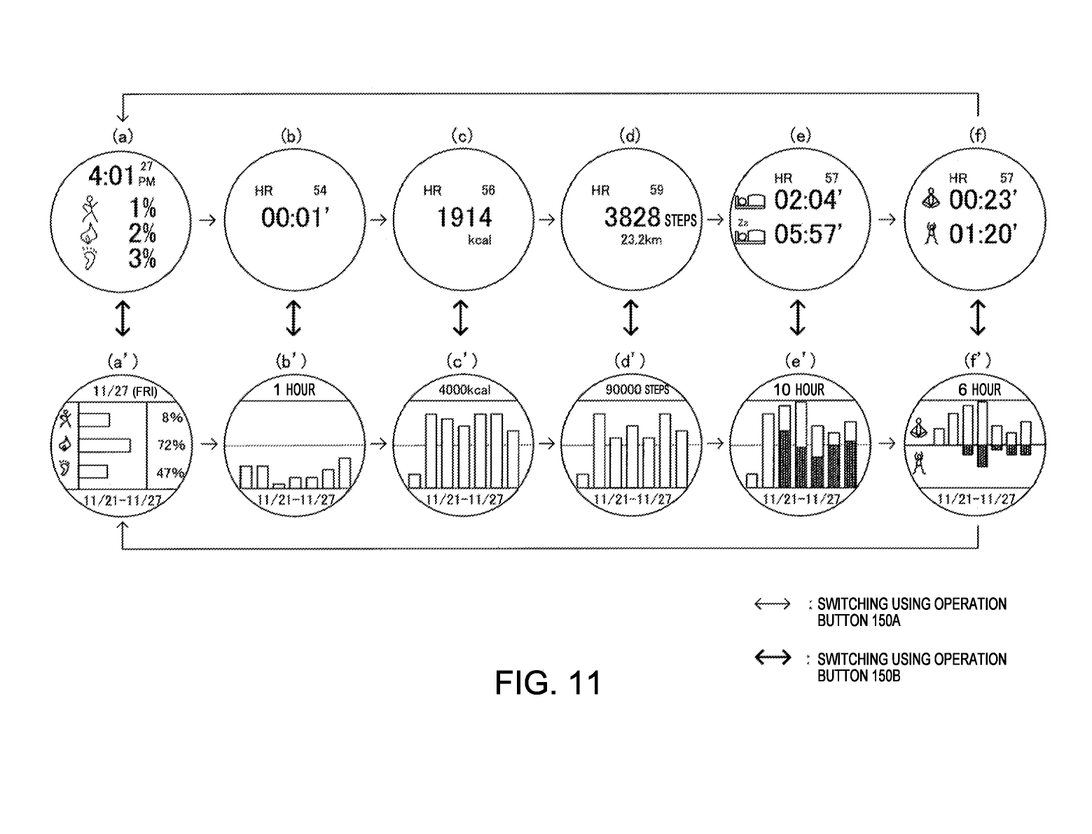

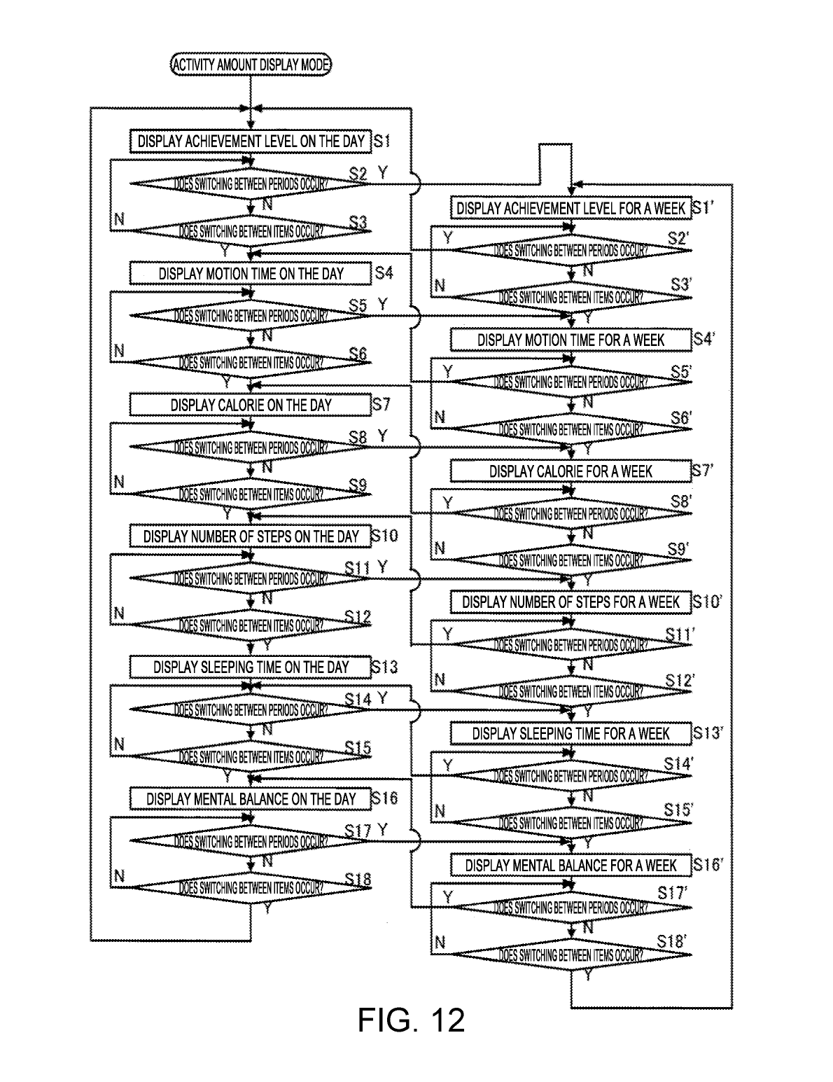

When the electronic apparatus 1 is in the time display mode (FIG. 1), for example, the user may press the operation button 150A once so as to cause the electronic apparatus 1 to transition to an activity amount display mode. Here, it is assumed that, as screens in the activity amount display mode, twelve display screens (refer to FIGS. 11(a) to 11(f′) which will be described later) are prepared. Outlines of the display screens (a) to (f′) are as follows.

(a) A display screen (hereinafter, referred to as an “achievement level screen on the day”) of an achievement level item (an example of a predetermined item) in a day (an example of a predetermined period, an example of a “different period”, and a first period) including the present time

(b) A display screen (hereinafter, referred to as a “motion time screen on the day”) of a motion time item (an example of a predetermined item) in a day including the present time

(c) A display screen (hereinafter, referred to as a “calorie consumption screen on the day”) of a calorie consumption item (an example of a predetermined item) in a day including the present time

(d) A display screen (hereinafter, referred to as a “number-of-steps screen on the day”) of a number-of-steps item (an example of a predetermined item) in a day including the present time

(e) A display screen (hereinafter, referred to as a “sleeping time screen on the day”) of a sleeping time item (an example of a predetermined item) in a day including the present time

(f) A display screen (hereinafter, referred to as a “mental balance screen on the day”) of a mental balance item (an example of a predetermined item) in a day including the present time

(a′) A display screen (hereinafter, referred to as an “achievement level screen for a week”) of an achievement level item for a week (an example of a period whose length is different from that of the predetermined period, and a second period) including the present time

(b′) A display screen (hereinafter, referred to as a “motion time screen for a week”) of a motion time item for a week including the present time

(c′) A display screen (hereinafter, referred to as a “calorie consumption screen for a week”) of a calorie consumption item for a week including the present time

(d′) A display screen (hereinafter, referred to as a “number-of-steps screen for a week”) of a number-of-steps item for a week including the present time

(e′) A display screen (hereinafter, referred to as a “sleeping time screen for a week”) of a sleeping time item for a week including the present time

(f′) A display screen (hereinafter, referred to as a “mental balance screen for a week”) of a mental balance item for a week including the present time

### 1-6-2. Achievement Level Screen

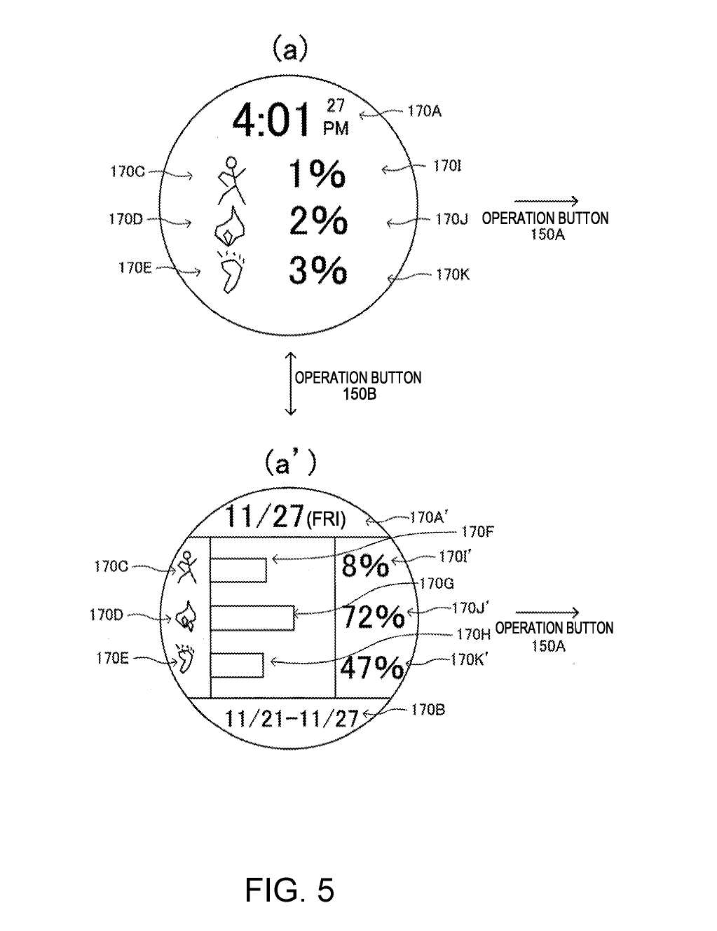

FIG. 5(a) is an enlarged view of an achievement level screen on the day, and FIG. 5(a′) is an enlarged view of an achievement level screen for a week.

As illustrated in FIG. 5(a), for example, an icon 170C (an icon of a lively body) of a motion time, and a text image 170I (“1%”) indicating a numerical value of an achievement level of a motion time in a day including the present time are disposed to be arranged on the achievement level screen on the day.

An icon 170D (an icon of flame) of calorie consumption, and a text image 170J (2%) indicating a numerical value of an achievement level of calorie consumption in a day including the present time are disposed to be arranged on the achievement level screen on the day.

An icon 170E (an icon of a footprint) of the number of steps, and a text image 170K (3%) indicating a numerical value of an achievement level of number of steps in a day including the present time are disposed to be arranged on the achievement level screen on the day.

A text image 170A indicating the present time may be disposed on the achievement level screen on the day.

Here, if the user presses (an example of a first operation, and an example of pressing a button once) the operation button 150B during display of the achievement level screen on the day (FIG. 5(a)), the achievement level screen on the day (FIG. 5(a)) is changed to the achievement level screen for a week (FIG. 5(a′)).

In other words, a function of switching between display object periods (period switching function) while maintaining a display object item may be assigned to the operation button 150B during display of the achievement level screen on the day (FIG. 5(a)).

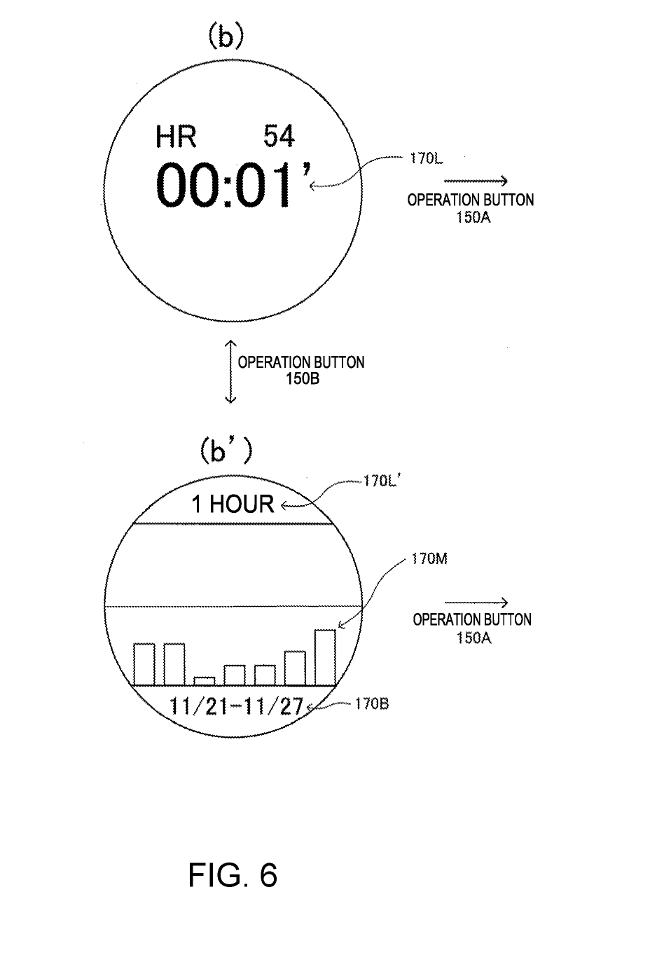

If the user presses the operation button 150A during display of the achievement level screen on the day (FIG. 5(a)), the achievement level screen on the day (FIG. 5(a)) is changed to a motion time screen on the day (FIG. 6(b)).

In other words, a function of switching between display object items (item switching function) while maintaining a display object period may be assigned to the operation button 150A during display of the achievement level screen on the day (FIG. 5(a)).

Next, as illustrated in FIG. 5(a′), the icon 170C (an icon of a lively body) of a motion time, a bar graph image 170F indicating a numerical value of an achievement level of a motion time for a week including the present time, and a text image 170I′ (“8%”) indicating a numerical value of an achievement level of a motion time for a week including the present time are disposed to be arranged on the achievement level screen for a week.

For example, the icon 170D (an icon of flame) of calorie consumption, a bar graph image 170G indicating a numerical value of an achievement level of calorie consumption for a week including the present time, and a text image 170J′ (“72%”) indicating a numerical value of an achievement level of calorie consumption for a week including the present time are disposed to be arranged on the achievement level screen for a week.

For example, the icon 170E (an icon of a footprint) of the number of steps, a bar graph image 170H indicating a numerical value of an achievement level of number of steps for a week including the present time, and a text image 170K′ (“47%”) indicating a numerical value of an achievement level of number of steps for a week including the present time are disposed to be arranged on the achievement level screen for a week.

A text image 170B (“11/21 to 11/27”) indicating a display object period (for a week) is disposed on the achievement level screen for a week.

A text image 170A′ indicating the present date and day of the week may be disposed on the achievement level screen for a week.

Here, if the user presses (an example of a second operation, an example of pressing a button once, and an example of the same operation as the first operation) the operation button 150B during display of the achievement level screen for a week (FIG. 5(a′)), the achievement level screen for a week (FIG. 5(a′)) is changed to the achievement level screen on the day (FIG. 5(a)).

In other words, a function of switching between display object periods (period switching function) while maintaining a display object item may be assigned to the operation button 150B during display of the achievement level screen for a week (FIG. 5(a′)).

If the user presses the operation button 150A during display of the achievement level screen for a week (FIG. 5(a′)), the achievement level screen for a week (FIG. 5(a′)) is changed to a motion time screen for a week (FIG. 6(b′)).

In other words, a function of switching between display object items (item switching function) while maintaining a display object period may be assigned to the operation button 150A during display of the achievement level screen for a week (FIG. 5(a′)).

### 1-6-3. Motion Time Screen

FIG. 6(b) is an enlarged view of a motion time screen on the day, and FIG. 6(b′) is an enlarged view of a motion time screen for a week.

As illustrated in FIG. 6(b), for example, a text image 170L (“00:01′”) indicating a numerical value of a motion time in a day including the present time is disposed on the motion time screen on the day.

Here, if the user presses (an example of a first operation) the operation button 150B during display of the motion time screen on the day (FIG. 6(b)), the motion time screen on the day (FIG. 6(b)) is changed to the motion time screen for a week (FIG. 6(b′)).

In other words, a function of switching between display object periods (period switching function) while maintaining a display object item may be assigned to the operation button 150B during display of the motion time screen on the day (FIG. 6(b)).

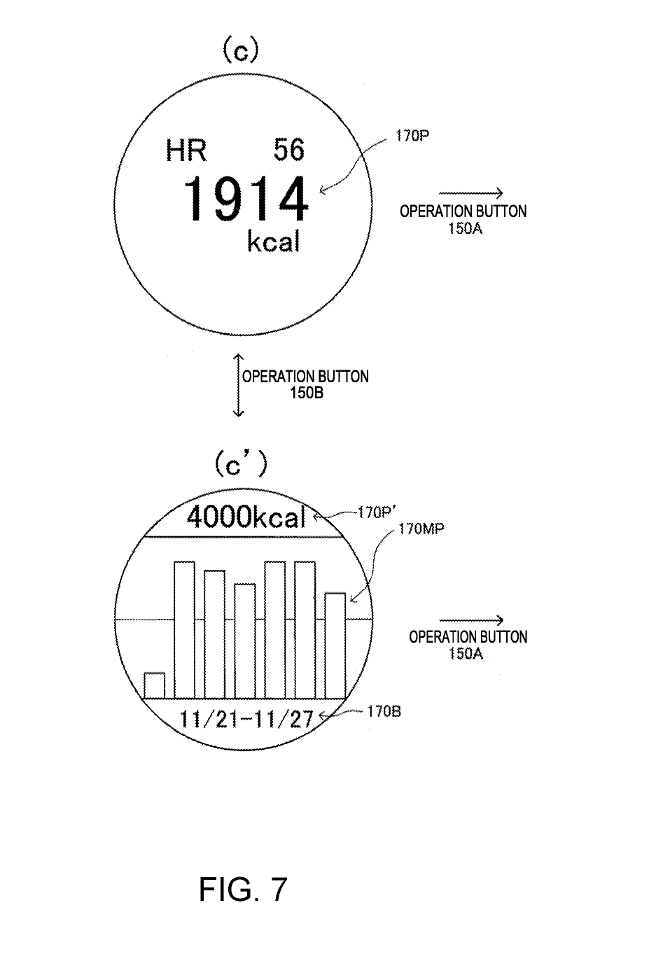

If the user presses the operation button 150A during display of the motion time screen on the day (FIG. 6(b)), the motion time screen on the day (FIG. 6(b)) is changed to a calorie consumption screen on the day (FIG. 7(c)).

In other words, a function of switching between display object items (item switching function) while maintaining a display object period may be assigned to the operation button 150A during display of the motion time screen on the day (FIG. 6(b)).

Next, as illustrated in FIG. 6(b′), a text image 170L′ (“one hour”) indicating a numerical value of a motion time screen for a week including the present time is disposed on the motion time screen for a week.

For example, a bar graph image 170M indicating a numerical value of a motion time every day for a week including the present time is disposed on the motion time screen for a week.

The text image 170B (“11/21 to 11/27”) indicating a display object period (for a week) is disposed on the motion time screen for a week.

Here, if the user presses (an example of a second operation) the operation button 150B during display of the motion time screen for a week (FIG. 6(b′)), the motion time screen for a week (FIG. 6(b′)) is changed to the motion time screen on the day (FIG. 6(b)).

In other words, a function of switching between display object periods (period switching function) while maintaining a display object item may be assigned to the operation button 150B during display of the motion time screen for a week (FIG. 6(b′)).

If the user presses the operation button 150A during display of the motion time screen for a week (FIG. 6(b′)), the motion time screen for a week (FIG. 6(b′)) is changed to a calorie consumption screen for a week (FIG. 7(c′)).

In other words, a function of switching between display object items (item switching function) while maintaining a display object period may be assigned to the operation button 150A during display of the motion time screen for a week (FIG. 6(b′)).

### 1-6-4. Calorie Consumption Screen

FIG. 7(c) is an enlarged view of a calorie consumption screen on the day, and FIG. 7(c′) is an enlarged view of a calorie consumption screen for a week.

As illustrated in FIG. 7(c), for example, a text image 170P (“1914 kcal′”) indicating a numerical value of calorie consumption in a day including the present time is disposed on the calorie consumption screen on the day.

Here, if the user presses (an example of a first operation) the operation button 150B during display of the calorie consumption screen on the day (FIG. 7(c)), the calorie consumption screen on the day (FIG. 7(c)) is changed to the calorie consumption screen for a week (FIG. 7(c′)).

In other words, a function of switching between display object periods (period switching function) while maintaining a display object item may be assigned to the operation button 150B during display of the calorie consumption screen on the day (FIG. 7(c)).

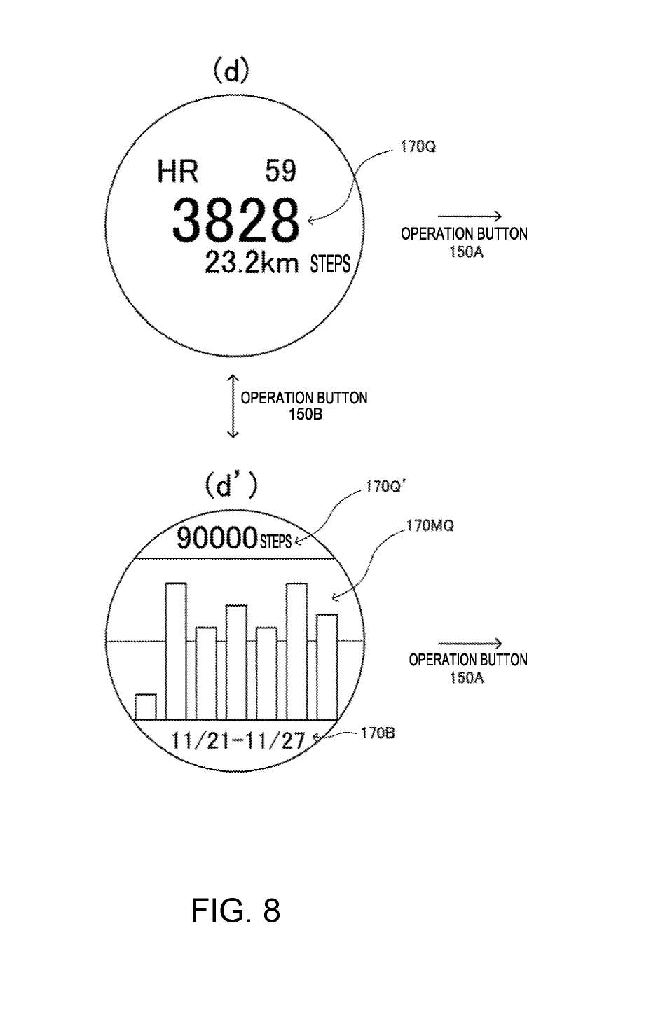

If the user presses the operation button 150A during display of the calorie consumption screen on the day (FIG. 7(c)), the calorie consumption screen on the day (FIG. 7(c)) is changed to a number-of-steps screen on the day (FIG. 8(d)).

In other words, a function of switching between display object items (item switching function) while maintaining a display object period may be assigned to the operation button 150A during display of the calorie consumption screen on the day (FIG. 7(c)).

Next, as illustrated in FIG. 7(c′), a text image 170P′ (“4000 kcal”) indicating a numerical value of calorie consumption for a week including the present time is disposed on the calorie consumption screen for a week.

For example, a bar graph image 170MP indicating a numerical value of calorie consumption every day for a week including the present time is disposed on the calorie consumption screen for a week.

The text image 170B (“11/21 to 11/27”) indicating a display object period (for a week) is disposed on the calorie consumption screen for a week.

Here, if the user presses (an example of a second operation) the operation button 150B during display of the calorie consumption screen for a week (FIG. 7(c′)), the calorie consumption screen for a week (FIG. 7(c′)) is changed to the calorie consumption screen on the day (FIG. 7(c)).

In other words, a function of switching between display object periods (period switching function) while maintaining a display object item may be assigned to the operation button 150B during display of the calorie consumption screen for a week (FIG. 7(c′)).

If the user presses the operation button 150A during display of the calorie consumption screen for a week (FIG. 7(c′)), the calorie consumption screen for a week (FIG. 7(c′)) is changed to a number-of-steps screen for a week (FIG. 8(d′)).

In other words, a function of switching between display object items (item switching function) while maintaining a display object period may be assigned to the operation button 150A during display of the calorie consumption screen for a week (FIG. 7(c′)).

### 1-6-5. Number-of-Steps Screen

FIG. 8(d) is an enlarged view of a number-of-steps screen on the day, and FIG. 8(d′) is an enlarged view of a number-of-steps screen for a week.

As illustrated in FIG. 8(d), for example, a text image 170Q (“3828 steps”) indicating a numerical value of the number of steps in a day including the present time is disposed on the number-of-steps screen on the day. FIG. 8(d) illustrates an example in which a text image (23.2 km) indicating a value of a movement distance along with the text image 170Q indicating the number of steps.

Here, if the user presses (an example of a first operation) the operation button 150B during display of the number-of-steps screen on the day (FIG. 8(d)), the number-of-steps screen on the day (FIG. 8(d)) is changed to the number-of-steps screen for a week (FIG. 8(d′)).

In other words, a function of switching between display object periods (period switching function) while maintaining a display object item may be assigned to the operation button 150B during display of the number-of-steps screen on the day (FIG. 8(d)).

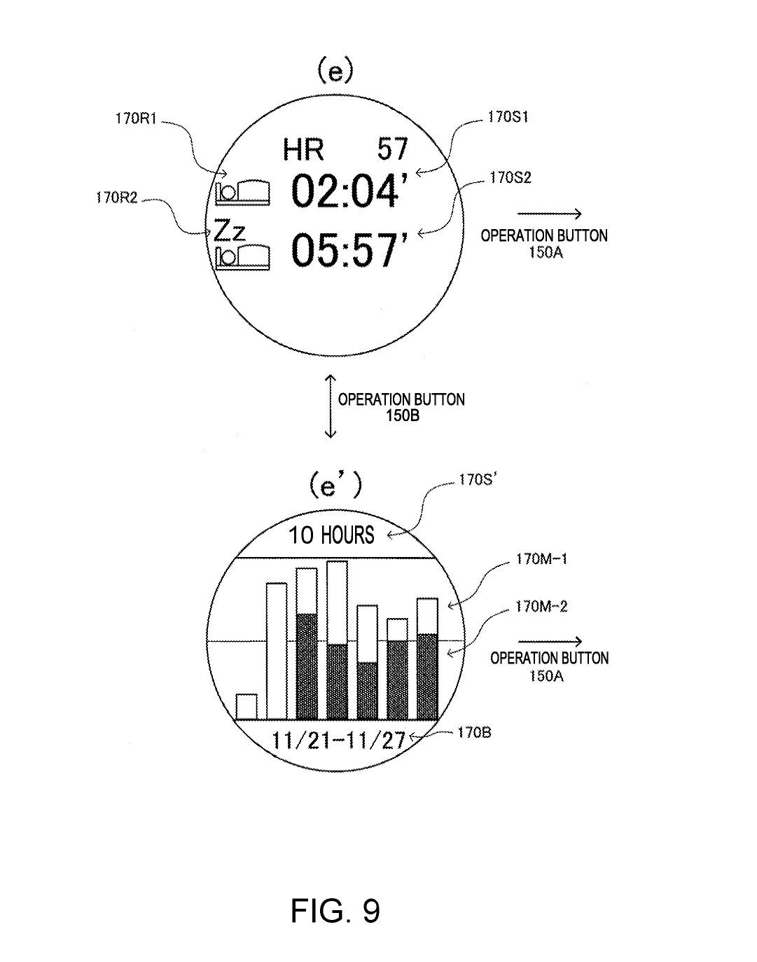

If the user presses the operation button 150A during display of the number-of-steps screen on the day (FIG. 8(d)), the number-of-steps screen on the day (FIG. 8(d)) is changed to a sleeping time screen on the day (FIG. 9(e)).

In other words, a function of switching between display object items (item switching function) while maintaining a display object period may be assigned to the operation button 150A during display of the number-of-steps screen on the day (FIG. 8(d)).

Next, as illustrated in FIG. 8(d′), a text image 170Q′ (“90000 steps”) indicating a numerical value of the number of steps for a week including the present time is disposed on the number-of-steps screen for a week.

For example, a bar graph image 170MQ indicating a numerical value of the number of steps every day for a week including the present time is disposed on the number-of-steps screen for a week.

The text image 170B (“11/21 to 11/27”) indicating a display object period (for a week) is disposed on the number-of-steps screen for a week.

Here, if the user presses (an example of a second operation) the operation button 150B during display of the number-of-steps screen for a week (FIG. 8(d′)), the number-of-steps screen for a week (FIG. 8(d′)) is changed to the number-of-steps screen on the day (FIG. 8(d)).

In other words, a function of switching between display object periods (period switching function) while maintaining a display object item may be assigned to the operation button 150B during display of the number-of-steps screen for a week (FIG. 8(d′)).

If the user presses the operation button 150A during display of the number-of-steps screen for a week (FIG. 8(d′)), the number-of-steps screen for a week (FIG. 8(d′)) is changed to a sleeping time screen for a week (FIG. 9(e′)).

In other words, a function of switching between display object items (item switching function) while maintaining a display object period may be assigned to the operation button 150A during display of the number-of-steps screen for a week (FIG. 8(d′)).

### 1-6-6. Sleeping Time Screen

FIG. 9(e) is an enlarged view of a sleeping time screen on the day, and FIG. 9(e′) is an enlarged view of a sleeping time screen for a week.

As illustrated in FIG. 9(e), for example, an icon 170R1 (an image of a bed) of a light sleeping time, and a text image 170S1 (“02:04′”) indicating a numerical value of a light sleeping time in a day including the present time are disposed to be arranged on the sleeping time screen on the day.

For example, an icon 170R2 (an image of a bed and “Zz”) of a deep sleeping time, and a text image 170S2 (“05:57”) indicating a numerical value of a deep sleeping time in a day including the present time are disposed to be arranged on the sleeping time screen on the day.

Here, if the user presses (an example of a first operation) the operation button 150B during display of the sleeping time screen on the day (FIG. 9(e)), the sleeping time screen on the day (FIG. 9(e)) is changed to the sleeping time screen for a week (FIG. 9(e′)).

In other words, a function of switching between display object periods (period switching function) while maintaining a display object item may be assigned to the operation button 150B during display of the sleeping time screen on the day (FIG. 9(e)).

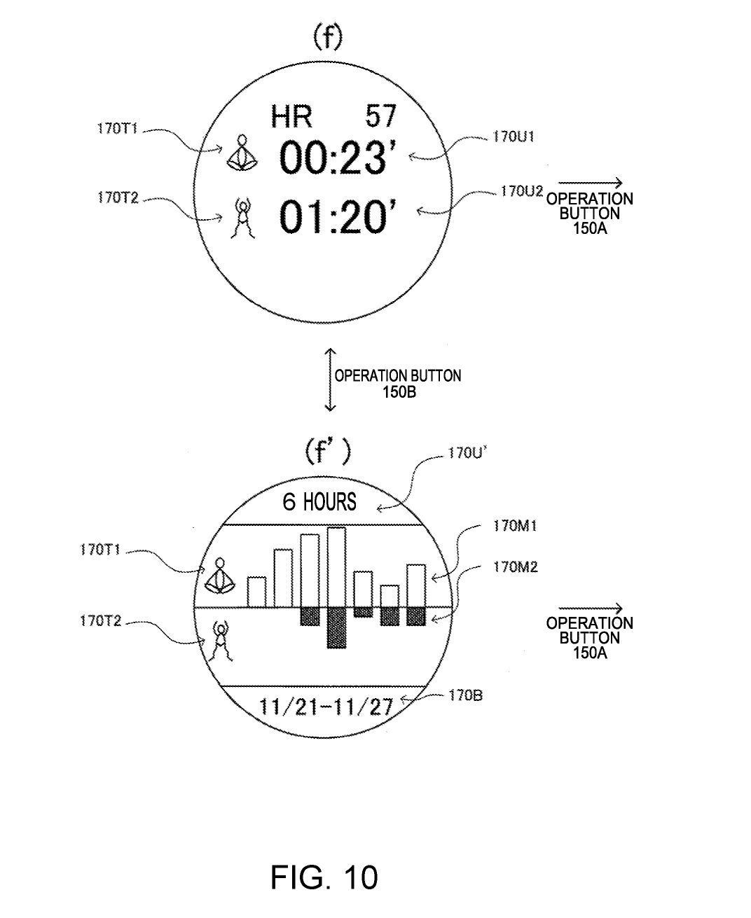

If the user presses the operation button 150A during display of the sleeping time screen on the day (FIG. 9(e)), the sleeping time screen on the day (FIG. 9(e)) is changed to a mental balance screen on the day (FIG. 10(f)).

In other words, a function of switching between display object items (item switching function) while maintaining a display object period may be assigned to the operation button 150A during display of the sleeping time screen on the day (FIG. 9(e)).

Next, as illustrated in FIG. 9(e′), a text image 170S′ (“ten hours”) indicating a numerical value of sleeping times for a week including the present time is disposed on the sleeping time screen for a week.

For example, a bar graph image 170M-1 indicating a numerical value of a light sleeping time every day for a week including the present time is disposed on the sleeping time screen for a week.

For example, a bar graph image 170M-2 indicating a numerical value of a deep sleeping time every day for a week including the present time is disposed on the sleeping time screen for a week.

In FIG. 9, the bar graph image 170M-1 and the bar graph image 170M-2 indicating sleeping times on the same day are displayed in different regions of the same block. In FIG. 9, in the same block, the region whose density is high is the bar graph image 170M-2 for a deep sleeping time, and the region whose density is low is the bar graph image 170M-1 for a light sleeping time.

The text image 170B (“11/21 to 11/27”) indicating a display object period (for a week) is disposed on the sleeping time screen for a week.

Here, if the user presses (an example of a second operation) the operation button 150B during display of the sleeping time screen for a week (FIG. 9(e′)), the sleeping time screen for a week (FIG. 9(e′)) is changed to the sleeping time screen on the day (FIG. 9(e)).

In other words, a function of switching between display object periods (period switching function) while maintaining a display object item may be assigned to the operation button 150B during display of the sleeping time screen for a week (FIG. 9(e′)).

If the user presses the operation button 150A during display of the sleeping time screen for a week (FIG. 9(e′)), the sleeping time screen for a week (FIG. 9(e′)) is changed to a mental balance screen for a week (FIG. 10(f′)).

In other words, a function of switching between display object items (item switching function) while maintaining a display object period may be assigned to the operation button 150A during display of the sleeping time screen for a week (FIG. 9(e′)).

### 1-6-7. Mental Balance Screen

FIG. 10(f) is an enlarged view of a mental balance screen on the day, and FIG. 10(f′) is an enlarged view of a mental balance screen for a week.

As illustrated in FIG. 10(f), for example, an icon 170T1 of a relaxing time, and a text image 170U1 (“00:23′”) indicating a numerical value of a relaxing time in a day including the present time are disposed to be arranged on the mental balance screen on the day.

For example, an icon 170T2 of an excitation time, and a text image 170U2 (“01:20′”) indicating a numerical value of an excitation time in a day including the present time are disposed to be arranged on the mental balance screen on the day.

Here, if the user presses (an example of a first operation) the operation button 150B during display of the mental balance screen on the day (FIG. 10(f)), the mental balance screen on the day (FIG. 10(f)) is changed to the mental balance screen for a week (FIG. 10(f′)).

In other words, a function of switching between display object periods (period switching function) while maintaining a display object item may be assigned to the operation button 150B during display of the mental balance screen on the day (FIG. 10(f)).

If the user presses the operation button 150A during display of the mental balance screen on the day (FIG. 10(f)), the mental balance screen on the day (FIG. 10(f)) is changed to the achievement level screen on the day (FIG. 5(a)).

In other words, a function of switching between display object items (item switching function) while maintaining a display object period may be assigned to the operation button 150A during display of the mental balance screen on the day (FIG. 10(f)).

Next, as illustrated in FIG. 10(f′), a text image 170U′ indicating a numerical value of relaxing times for a week including the present time is disposed on the mental balance screen for a week.

For example, the icon 170T1 of a relaxing time and a bar graph image 170M1 indicating a numerical value of a relaxing time every day for a week including the present time are disposed to be arranged on the mental balance screen for a week.

For example, the icon 170T2 of an excitation time and a bar graph image 170M2 indicating a numerical value of an excitation time every day for a week including the present time are disposed to be arranged on the mental balance screen for a week.

In FIG. 10(f′), the bar graph images 170M1 and 170M2 on the same day are displayed in different regions of the same block. In FIG. 10, in the same block, the region whose density is low is the bar graph image 170M1 for a relaxing time, and the region whose density is high is the bar graph image 170M2 for an excitation time.

The text image 170B (“11/21 to 11/27”) indicating a display object period (for a week) is disposed on the mental balance screen for a week.

Here, if the user presses (an example of a second operation) the operation button 150B during display of the mental balance screen for a week (FIG. 10(f′)), the mental balance screen for a week (FIG. 10(f′)) is changed to the mental balance screen on the day (FIG. 10(f)).

In other words, a function of switching between display object periods (period switching function) while maintaining a display object item may be assigned to the operation button 150B during display of the mental balance screen for a week (FIG. 10(f′)).

If the user presses the operation button 150A during display of the mental balance screen for a week (FIG. 10(f′)), the mental balance screen for a week (FIG. 10(f′)) is changed to the achievement level screen for a week (FIG. 5(a′)).

In other words, a function of switching between display object items (item switching function) while maintaining a display object period may be assigned to the operation button 150A during display of the mental balance screen for a week (FIG. 10(f′)).

### 1-7. Screen Switching Pattern

FIG. 11 is a diagram illustrating a screen switching pattern in the activity amount display mode.

FIG. 11(a) illustrates the achievement level screen on the day; FIG. 11(b) illustrates the motion time screen on the day; FIG. 11(c) illustrates the calorie consumption screen on the day; FIG. 11(d) illustrates the number-of-steps screen on the day; FIG. 11(e) illustrates the sleeping time screen on the day; and FIG. 11(f) illustrates the mental balance screen on the day. FIG. 11(a′) illustrates the achievement level screen for a week; FIG. 11(b′) illustrates the motion time screen for a week; FIG. 11(c′) illustrates the calorie consumption screen for a week; FIG. 11(d′) illustrates the number-of-steps screen for a week; FIG. 11(e′) illustrates the sleeping time screen for a week; and FIG. 11(f′) illustrates the mental balance screen for a week.

In FIG. 11, a thick arrow indicates a screen switching direction when a pressing operation (an example of a first operation or a second operation) is performed on the operation button 150B (here, the period switching function is assigned thereto), and a thin arrow indicates a screen switching direction when a pressing operation (here, the item switching function is assigned thereto) is performed on the operation button 150A.

As illustrated in FIG. 11, the user presses the operation button 150B (that is, an operation button to which the period switching function is assigned) when the electronic apparatus 1 is in the activity amount display mode, and can thus switch a display object period between “the day” and “a week” while a display object item is maintained.

As illustrated in FIG. 11, the user repeatedly presses the operation button 150A (that is, an operation button to which the item switching function is assigned) when a display object period is “the day” or “a week”, and can thus (cyclically) switch a display object item among the “achievement level item”, the “motion time item”, the “calorie consumption item”, the “number-of-steps item”, the “sleeping time item”, and the “mental balance item” while the display object period is maintained.

The user's action required to change the display object period is a single action (one touch operation) such as pressing of the operation button, the user's action required to change the display object item is a single action (one touch operation) such as pressing of the operation button.

Therefore, the electronic apparatus 1 is convenient to use for a user who positively compares pieces of activity amount data in different periods or positively compares pieces of activity amount data of different items.

### 1-8. Flow in Activity Amount Display Mode

FIG. 12 is a flowchart illustrating a process (an example of a display method) in the processing section 120 of the electronic apparatus 1 in the activity amount display mode. FIG. 12 is summarized as follows.

Steps S1 to S3 in FIG. 12 correspond to processes regarding the achievement level screen on the day.

Steps S1′ to S3′ in FIG. 12 correspond to processes regarding the achievement level screen for a week.

Steps S4 to S6 in FIG. 12 correspond to processes regarding the motion time screen on the day.

Steps S4′ to S6′ in FIG. 12 correspond to processes regarding the motion time screen for a week.

Steps S7 to S9 in FIG. 12 correspond to processes regarding the calorie consumption screen on the day.

Steps S7′ to S9′ in FIG. 12 correspond to processes regarding the calorie consumption screen for a week.

Steps S10 to S12 in FIG. 12 correspond to processes regarding the number-of-steps screen on the day.

Steps S10′ to S12′ in FIG. 12 correspond to processes regarding the number-of-steps screen for a week.

Steps S13 to S15 in FIG. 12 correspond to processes regarding the sleeping time screen on the day.

Steps S13′ to S15′ in FIG. 12 correspond to processes regarding the sleeping time screen for a week.

Steps S16 to S18 in FIG. 12 correspond to processes regarding the mental balance screen on the day.

Steps S16′ to S18′ in FIG. 12 correspond to processes regarding the mental balance screen for a week.

### 1-8-1. Processes Regarding Achievement Level Screen on the Day

Details of the processes (steps S1 to S3) regarding the achievement level screen on the day are as follows.

The processing section 120 starts a display process (step S1) of the achievement level screen on the day, and repeatedly performs a process (step S2) of determining whether or not the operation button 150B (that is, an operation button to which the period switching function is assigned) is pressed and a process (step S3) of determining whether or not the operation button 150A (that is, an operation button to which the item switching function is assigned) is pressed during display of the achievement level screen on the day (N in step S2 and N in step S3). If it is determined that the operation button 150B (that is, an operation button to which the period switching function is assigned) is pressed (Y in step S2), the processing section 120 transitions to a display process (step S1′) of a screen (here, the achievement level screen for a week) having a differing display object period. If it is determined that the operation button 150A (that is, an operation button to which the item switching function is assigned) is pressed (Y in step S3), the processing section 120 transitions to a display process (step S4) of a screen (here, the motion time screen on the day) having a differing display object item.

### 1-8-2. Processes Regarding Achievement Level Screen for a Week

Details of the processes (steps S1′ to S3′) regarding the achievement level screen for a week are as follows.

The processing section 120 starts a display process (step S1′) of the achievement level screen for a week, and repeatedly performs a process (step S2′) of determining whether or not the operation button 150B (that is, an operation button to which the period switching function is assigned) is pressed and a process (step S3′) of determining whether or not the operation button 150A (that is, an operation button to which the item switching function is assigned) is pressed during display of the achievement level screen for a week (N in step S2′ and N in step S3′). If it is determined that the operation button 150B (that is, an operation button to which the period switching function is assigned) is pressed (Y in step S2′), the processing section 120 transitions the display process (step S1) of a screen (here, the achievement level screen on the day) having a differing display object period. If it is determined that the operation button 150A (that is, an operation button to which the item switching function is assigned) is pressed (Y in step S3′), the processing section 120 transitions to a display process (step S4′) of a screen (here, the motion time screen for a week) having a differing display object item.

### 1-8-3. Processes Regarding Motion Time Screen on the Day

Details of the processes (steps S4 to S6) regarding the motion time screen on the day are as follows.

The processing section 120 starts a display process (step S4) of the motion time screen on the day, and repeatedly performs a process (step S5) of determining whether or not the operation button 150B (that is, an operation button to which the period switching function is assigned) is pressed and a process (step S6) of determining whether or not the operation button 150A (that is, an operation button to which the item switching function is assigned) is pressed during display of the motion time screen on the day (N in step S5 and N in step S6). If it is determined that the operation button 150B (that is, an operation button to which the period switching function is assigned) is pressed (Y in step S5), the processing section 120 transitions to a display process (step S4′) of a screen (here, the motion time screen for a week) having a differing display object period. If it is determined that the operation button 150A (that is, an operation button to which the item switching function is assigned) is pressed (Y in step S6), the processing section 120 transitions to a display process (step S7) of a screen (here, the calorie consumption screen on the day) having a differing display object item.

### 1-8-4. Processes Regarding Motion Time Screen for a Week

Details of the processes (steps S4′ to S6′) regarding the motion time screen for a week are as follows.

The processing section 120 starts a display process (step S4′) of the motion time screen for a week, and repeatedly performs a process (step S5′) of determining whether or not the operation button 150B (that is, an operation button to which the period switching function is assigned) is pressed and a process (step S6′) of determining whether or not the operation button 150A (that is, an operation button to which the item switching function is assigned) is pressed during display of the motion time screen for a week (N in step S5′ and N in step S6′). If it is determined that the operation button 150B (that is, an operation button to which the period switching function is assigned) is pressed (Y in step S5′), the processing section 120 transitions to a display process (step S4) of a screen (here, the motion time screen on the day) having a differing display object period. If it is determined that the operation button 150A (that is, an operation button to which the item switching function is assigned) is pressed (Y in step S6′), the processing section 120 transitions to a display process (step S7′) of a screen (here, the calorie consumption screen for a week) having a differing display object item.

### 1-8-5. Processes Regarding Calorie Consumption Screen on the Day

Details of the processes (steps S7 to S9) regarding the calorie consumption screen on the day are as follows.

The processing section 120 starts a display process (step S7) of the calorie consumption screen on the day, and repeatedly performs a process (step S8) of determining whether or not the operation button 150B (that is, an operation button to which the period switching function is assigned) is pressed and a process (step S9) of determining whether or not the operation button 150A (that is, an operation button to which the item switching function is assigned) is pressed during display of the calorie consumption screen on the day (N in step S8 and N in step S9). If it is determined that the operation button 150B (that is, an operation button to which the period switching function is assigned) is pressed (Y in step S8), the processing section 120 transitions to a display process (step S7′) of a screen (here, the calorie consumption screen for a week) having a differing display object period. If it is determined that the operation button 150A (that is, an operation button to which the item switching function is assigned) is pressed (Y in step S9), the processing section 120 transitions to a display process (step S10) of a screen (here, the number-of-steps screen on the day) having a differing display object item.

### 1-8-6. Processes Regarding Calorie Consumption Screen for a Week

Details of the processes (steps S7′ to S9′) regarding the calorie consumption screen for a week are as follows.

The processing section 120 starts a display process (step S7′) of the calorie consumption screen for a week, and repeatedly performs a process (step S8′) of determining whether or not the operation button 150B (that is, an operation button to which the period switching function is assigned) is pressed and a process (step S9′) of determining whether or not the operation button 150A (that is, an operation button to which the item switching function is assigned) is pressed during display of the calorie consumption screen for a week (N in step S8′ and N in step S9′). If it is determined that the operation button 150B (that is, an operation button to which the period switching function is assigned) is pressed (Y in step S8′), the processing section 120 transitions to a display process (step S7) of a screen (here, the calorie consumption screen on the day) having a differing display object period. If it is determined that the operation button 150A (that is, an operation button to which the item switching function is assigned) is pressed (Y in step S9′), the processing section 120 transitions to a display process (step S10′) of a screen (here, the number-of-steps screen for a week) having a differing display object item.

### 1-8-7. Processes Regarding Number-Of-Steps Screen on the Day

Details of the processes (steps S10 to S12) regarding the number-of-steps screen on the day are as follows.

The processing section 120 starts a display process (step S10) of the number-of-steps screen on the day, and repeatedly performs a process (step S11) of determining whether or not the operation button 150B (that is, an operation button to which the period switching function is assigned) is pressed and a process (step S12) of determining whether or not the operation button 150A (that is, an operation button to which the item switching function is assigned) is pressed during display of the number-of-steps screen on the day (N in step S11 and N in step S12). If it is determined that the operation button 150B (that is, an operation button to which the period switching function is assigned) is pressed (Y in step S11), the processing section 120 transitions to a display process (step S10′) of a screen (here, the number-of-steps screen for a week) having a differing display object period. If it is determined that the operation button 150A (that is, an operation button to which the item switching function is assigned) is pressed (Y in step S12), the processing section 120 transitions to a display process (step S13) of a screen (here, the sleeping time screen on the day) having a differing display object item.

### 1-8-8. Processes Regarding Number-of-Steps Screen for a Week

Details of the processes (steps S10′ to S12′) regarding the number-of-steps screen for a week are as follows.

The processing section 120 starts a display process (step S10′) of the number-of-steps screen for a week, and repeatedly performs a process (step S11′) of determining whether or not the operation button 150B (that is, an operation button to which the period switching function is assigned) is pressed and a process (step S12′) of determining whether or not the operation button 150A (that is, an operation button to which the item switching function is assigned) is pressed during display of the number-of-steps screen for a week (N in step S11′ and N in step S12′). If it is determined that the operation button 150B (that is, an operation button to which the period switching function is assigned) is pressed (Y in step S11′), the processing section 120 transitions to a display process (step S10) of a screen (here, the number-of-steps screen on the day) having a differing display object period. If it is determined that the operation button 150A (that is, an operation button to which the item switching function is assigned) is pressed (Y in step S12′), the processing section 120 transitions to a display process (step S13′) of a screen (here, the sleeping time screen for a week) having a differing display object item.

### 1-8-9. Processes Regarding Sleeping Time Screen on the Day

Details of the processes (steps S13 to S15) regarding the sleeping time screen on the day are as follows.

The processing section 120 starts a display process (step S13) of the sleeping time screen on the day, and repeatedly performs a process (step S14) of determining whether or not the operation button 150B (that is, an operation button to which the period switching function is assigned) is pressed and a process (step S15) of determining whether or not the operation button 150A (that is, an operation button to which the period switching function is assigned) is pressed during display of the sleeping time screen on the day (N in step S14 and N in step S15). If it is determined that the operation button 150B (that is, an operation button to which the period switching function is assigned) is pressed (Y in step S14), the processing section 120 transitions to a display process (step S13′) of a screen (here, the sleeping time screen for a week) having a differing display object period. If it is determined that the operation button 150A (that is, an operation button to which the item switching function is assigned) is pressed (Y in step S15), the processing section 120 transitions to a display process (step S16) of a screen (here, the mental balance screen on the day) having a differing display object item.

### 1-8-10. Processes Regarding Sleeping Time Screen for a Week

Details of the processes (steps S13′ to S15′) regarding the sleeping time screen for a week are as follows.

The processing section 120 starts a display process (step S13′) of the sleeping time screen for a week, and repeatedly performs a process (step S14′) of determining whether or not the operation button 150B (that is, an operation button to which the period switching function is assigned) is pressed and a process (step S15′) of determining whether or not the operation button 150A (that is, an operation button to which the item switching function is assigned) is pressed during display of the sleeping time screen for a week (N in step S14′ and N in step S15′). If it is determined that the operation button 150B (that is, an operation button to which the period switching function is assigned) is pressed (Y in step S14′), the processing section 120 transitions to a display process (step S13) of a screen (here, the sleeping time screen on the day) having a differing display object period. If it is determined that the operation button 150A (that is, an operation button to which the item switching function is assigned) is pressed (Y in step S15′), the processing section 120 transitions to a display process (step S16′) of a screen (here, the mental balance screen for a week) having a differing display object item.

### 1-8-11. Processes Regarding Mental Balance Screen on the Day

Details of the processes (steps S16 to S18) regarding the mental balance screen on the day are as follows.

The processing section 120 starts a display process (step S16) of the mental balance screen on the day, and repeatedly performs a process (step S17) of determining whether or not the operation button 150B (that is, an operation button to which the period switching function is assigned) is pressed and a process (step S18) of determining whether or not the operation button 150A (that is, an operation button to which the item switching function is assigned) is pressed during display of the mental balance screen on the day (N in step S17 and N in step S18). If it is determined that the operation button 150B (that is, an operation button to which the period switching function is assigned) is pressed (Y in step S17), the processing section 120 transitions to a display process (step S16′) of a screen (here, the mental balance screen for a week) having a differing display object period. If it is determined that the operation button 150A (that is, an operation button to which the item switching function is assigned) is pressed (Y in step S18), the processing section 120 transitions to a display process (step S1) of a screen (here, the achievement level screen on the day) having a differing display object item.

### 1-8-12. Processes Regarding Mental Balance Screen for a Week

Details of the processes (steps S16′ to S18′) regarding the mental balance screen for a week are as follows.

The processing section 120 starts a display process (step S16′) of the mental balance screen for a week, and repeatedly performs a process (step S17′) of determining whether or not the operation button 150B (that is, an operation button to which the period switching function is assigned) is pressed and a process (step S18′) of determining whether or not the operation button 150A (that is, an operation button to which the item switching function is assigned) is pressed during display of the mental balance screen for a week (N in step S17′ and N in step S18′). If it is determined that the operation button 150B (that is, an operation button to which the period switching function is assigned) is pressed (Y in step S17′), the processing section 120 transitions to a display process (step S16) of a screen (here, the mental balance screen on the day) having a differing display object period. If it is determined that the operation button 150A (that is, an operation button to which the item switching function is assigned) is pressed (Y in step S18′), the processing section 120 transitions to a display process (step S1′) of a screen (here, the achievement level screen for a week) having a differing display object item.

The steps illustrated in FIG. 12 may be replaced within an allowable range. For example, a determination of period switching and a determination of item switching may be performed in parallel to each other. Although not illustrated in FIG. 12, in a case where an instruction for switching to another mode (for example, the time display mode) is input from the user during execution of the flow illustrated in FIG. 12, the processing section 120 suspends the flow illustrated in FIG. 12, and starts a process in another mode.

### 2. Operations and Effects of Embodiment

As described above, the electronic apparatus 1 according to the present embodiment includes the operation button 150B that receives an input operation from a user; the sensors 110, 111, . . . , and 116 that perform detection related to an amount of activity of the user; the display section 170 that displays activity amount data by using outputs from the sensors 110, 111, . . . , and 116; and the processing section 120 that maintains a display object item of the activity amount data to be the item and changes a display object period of the activity amount data to a week in a case where an operation of pressing the operation button 150B is received (Y in step S2, Y in step S5, Y in step S8, Y in step S11, Y in step S14, and Y in step S17) when the activity amount data on the day regarding any one of the achievement level item, the motion time item, the number-of-steps item, the calorie consumption item, the sleeping time item, and the mental balance item is displayed.

The processing section 120 maintains a display object item of the activity amount data to be the item and changes a display object period of the activity amount data from a week to the day in a case where an operation of pressing the operation button 150B is received (Y in step S2′, Y in step S5′, Y in step S8′, Y in step S11′, Y in step S14′, and Y in step S17′) when the activity amount data for a week regarding the item is displayed.

The electronic apparatus 1 is portable, and thus has restriction in a size of the display section unlike an installation type apparatus such as a personal computer. Thus, if a large amount of data is simultaneously displayed on the same screen, there is concern that it may be hard to view each piece of data. However, in a case where an operation of pressing the operation button 150B is received when activity amount data on the day regarding a certain item is displayed, the processing section 120 of the electronic apparatus 1 maintains a display object item of the activity amount data to be the item, and changes a display object period of the activity amount data from “the day” to “a week”. In this case, basically, if the user has only to perform an pressing operation on the operation button 150B, a display object period can be changed from “the day” to “a week” while maintaining a display object item of activity amount data. Therefore, the user can compare data on the day with data for a week with respect to the same item on the basis of a change of the display content before and after the pressing operation on the operation button 150B.

### 3. Modification Examples of Embodiment

### 3-1. Button Operation

The processing section 120 of the above-described embodiment assigns the item switching function to the operation button 150A, but may assign the item switching function to other operation buttons.

The processing section 120 of the above-described embodiment assigns the period switching function to the operation button 150B, but may assign the period switching function to other buttons.

The processing section 120 of the above-described embodiment assigns the period switching function to the single operation button 150B, but may assign a switching function from “the day” to “a week” and a switching function from “a week” to “the day” to other operation buttons. The user can easily understand which period is selected as a display object period.

The processing section 120 of the above-described embodiment regards a pressing operation on the operation button 150B performed by the user as a period switching instruction, but, in order to differentiate a period switching instruction from a lighting instruction, a long pressing operation on the operation button 150B performed by the user may be regarded as the lighting instruction, and a short pressing operation on the operation button 150B performed by the user may be regarded as the period switching instruction. Alternatively, the processing section 120 of the above-described embodiment may regard a short pressing operation on the operation button 150B performed by the user as the lighting instruction, and regard a long pressing operation on the operation button 150B performed by the user as the period switching instruction.

The processing section 120 of the above-described embodiment assigns the item switching function to a specific operation button (operation button 150A) and assigns the period switching function to a specific operation button (operation button 150B), but may assign a function of setting a display object period to a week and the item switching function (a function of a one-week button) to a specific operation button (for example, the operation button 150C) and may assign a function of setting a display object period to the day and the item switching function (a function of a the-day button) to a specific operation button (for example, the operation button 150D). If such a function is assigned to a dedicated button, it is possible to realize a simpler configuration which can be easily understood by a user.

In this case, if the user presses the operation button 150C (one-week button) when a display object period is “the day”, a display object period can be changed to “a week” while a display object item is maintained, and if the user presses the operation button 150D (the-day button) when a display object period is “a week”, a display object period can be changed to “the day” while a display object item is maintained.

When a display object period is “the day”, the user presses the operation button 150D (the-day button) and can thus (cyclically) switch a display object item among the “achievement level”, the “motion time item”, the “calorie consumption item”, the “number-of-steps item”, the “sleeping time item”, and the “mental balance item” while a display object period is maintained. When a display object period is “a week”, the user presses the operation button 150C (one-week button) and can thus (cyclically) switch a display object item among the “achievement level”, the “motion time item”, the “calorie consumption item”, the “number-of-steps item”, the “sleeping time item”, and the “mental balance item” while a display object period is maintained.

Among functions assigned to the respective operation buttons 150A, 150B, 150C and 150D by the processing section 120 of the above-described embodiment, at least one of lighting and putting-out of a light source, a measurement (measurement of motion) starting instruction and a measurement finishing instruction of the split time or the like, and a lap time recording instruction may be omitted. In a case where the number of assigned functions is small, if the period switching function is assigned to a dedicated button, it is possible to realize a simpler configuration which can be easily understood by a user.

The processing section 120 of the above-described embodiment regards “one-pressing” of a predetermined button performed by a user as a period switching instruction, but may regard “two-pressing” of the button as the period switching instruction. The processing section 120 of the above-described embodiment regards “one-pressing” of a predetermined button performed by a user as an item switching instruction, but may regard “two-pressing” of the button as the item switching instruction. Similarly, “long pressing”, “short pressing”, “three-pressing”, or the like may be regarded as a specific instruction. The two-pressing mentioned here indicates that a pressing operation is continuously performed twice within a predetermined period, and the three-pressing indicates that a pressing operation is continuously performed three times within a predetermined period (each operation such as one-pressing, two-pressing, long pressing, and short pressing performed by a user is an example of a single action).

### 3-2. Graph Display

The processing section 120 of the above-described embodiment displays a change in data in a display object period by using a bar graph with respect to at least one item, but may display the change by using graphs of other types such as a line graph.

For example, the electronic apparatus 1 may display a change of data on the day regarding a certain item by using, for example, a line graph every hour, and may display a change of data for a week regarding the same item by using, for example, a line graph every day.

The processing section 120 of the above-described embodiment displays data regarding at least one item with a numerical value image, but numerical value images may be changed in a time series by a user continuously pressing (or repeatedly pressing) a predetermined operation button (which is different from the operation buttons 150A and 150B). Alternatively, the processing section 120 of the above-described embodiment may list a plurality of numerical value images on a screen so that changes of numerical values are displayed in a list form.

The processing section 120 of the above-described embodiment may display data regarding at least one item along with a target for the item (a target and a result). In this case, the processing section 120 may highlight data in a case where data regarding a certain item reaches a target. The highlighting of data is performed by changing at least one of contrast, brightness, a color, and saturation of the data, displaying the data in an inverting manner, displaying the data in a blinking manner, adding a mark thereto, or enlarging a display size of the data.

The processing section 120 of the above-described embodiment may arrange and display at least one combination of (i) a target and a result, (ii) a result and an expectation, and (iii) the maximum, the minimum, and an average.

### 3-3. Display Object Period

The processing section 120 of the above-described embodiment sets period switching in order of “the day” and “a week”, but may set period switch in order of “a week” and “the day”, and may allow a user to set the order (customizable).

The processing section 120 of the above-described embodiment uses “the day” and “a week” as two display object periods between which switching can occur, but there is no limitation to two periods, and may allow a user to select a display object period (customizable).

For example, the processing section 120 of the above-described embodiment may add, as one of display object periods between which switching can occur, at least one of “an hour including the present time”, “two hours including the present time”, “three hours including the present time”, “six hours including the present time”, “twelve hours including the present time”, “twenty-four hours including the present time”, “a week including the present time”, “a month including the present time”, “a year including the present time”, “three years including the present time”, “five years including the present time” . . . .

For example, the processing section 120 of the above-described embodiment may add, as one of display object periods between which switching can occur, at least one of “the day”, “yesterday”, “the day before yesterday”, . . . , “this week”, “last week”, “the week before last”, . . . , “this month”, “last month”, “the month before last”, . . . , “this year”, “last year”, “the year before last”, . . . .

### 3-4. Display Object Item

The processing section 120 of the above-described embodiment displays the achievement level screen on the day (FIG. 5(a)) at the beginning of the activity amount display mode, but may display a time display screen (FIG. 1) instead of the achievement level screen on the day (FIG. 5(a)).

The processing section 120 of the above-described embodiment sets item switching in order of (a) an achievement level, (b) a motion time, (c) calorie consumption, (d) the number of steps, (e) a sleeping time, and (f) mental balance, but may set item switching in differing order, and may allow a user to set the order (customizable).

The processing section 120 of the above-described embodiment uses (a) an achievement level, (b) a motion time, (c) calorie consumption, (d) the number of steps, (e) a sleeping time, and (f) mental balance as six display object items among which switching can occur, but may exclude some of the six items from the display object items, may add other items to the display object items, and may allow a user to select a display object item (customizable).

For example, the processing section 120 of the above-described embodiment may add at least one of (g) the maximum oxygen intake (VO₂max) and (h) the minimum oxygen intake (VO₂ min), as one of display object items among which switching can occur.

Here, the maximum oxygen intake (VO₂max) is the maximum oxygen intake of a user in an object period.

The minimum oxygen intake (VO₂ min) is the minimum oxygen intake of a user in an object period. The oxygen intake may be calculated (estimated) according to a predetermined calculation formula by using parameters such as the age, the sex, and a weight of a user. The calculation formula is well known, and thus a description thereof will be omitted here.

For example, the processing section 120 of the above-described embodiment may add at least one of (i) the maximum pulse rate and (j) the minimum pulse rate, as one of display object items among which switching can occur.

Here, the maximum pulse rate “HRmax” is the maximum pulse rate of a user in a display object period, and may be calculated according to, for example, “HRmax=220−the age of a subject”.

The minimum pulse rate “HRmin” is the minimum pulse rate of a user in an object period, and may be set to, for example, a pulse rate during relaxing. A pulse rate during relaxing of a general adult is about “60 to 70”. A pulse rate during relaxing may be determined in this range on the basis of the sex or the age of a user, so as to be set as the minimum pulse rate “HRmin”.

### 3-5. Examples of Activity Amount Items

The processing section 120 of the above-described embodiment may add at least one of the following items as one of display object items among which switching can occur.

Examples of activity amount items: a movement distance, a motion time, the number of steps, a walking pace, a walking pitch, a stride, the number of fast steps, the number of running steps, the number of increasing stories (“five stories”, “two stories”, or the like), the number of increasing stairs (“100 stairs”, “200 stairs”, or the like), a heart rate, oxygen intake, an amount of perspiration, water intake (which is manually input by a user), calorie consumption, calorie intake (which is manually input by a user), calories balance, a weight (which is input through communication with a weight meter or is manually input by a user), a waist size (which is manually input by a user), a sleeping time, sleep quality (distinction between deep and light sleeps, distinction between good and bad sleeps, a score, and the like), balance between sleep quality (a ratio between a deep sleeping time and a light sleeping time), balance between an excitation time and a relaxing time (mental balance), a target achievement level, an ultraviolet quantity, . . . , and the like.

### 3-6. Performance in Motion

The processing section 120 of the above-described embodiment may add at least one of the following items as one of display object items among which switching can occur. The following items are performance items in motion. Some of the performance items may overlap some of the activity amount items. The electronic apparatus 1 measuring or recording such motion performance may be said to be a motion performance monitoring apparatus.

Examples of performance items: a motion distance (a movement distance or an accumulated movement distance), a motion time, a motion time in a predetermined heartbeat zone, the number of steps, the number of lap steps, a running pace, a running pitch, a stride, the split time, the lap time, accumulated increasing altitude, accumulated decreasing altitude, an elevation (average altitude of a motion location), gradient, the number of times of training (the number of times of running or the like), a target achievement level, an attitude (running attitude), left/right difference, foot contact time, a directly-below landing ratio, propulsion efficiency, slow turnover of the legs, a landing brake quantity, landing impact, a heart rate, calorie consumption, oxygen intake, an amount of perspiration, water intake, an expected motion distance under a predetermined condition (a movement distance or an expected accumulated distance), the time until reaching a predetermined heartbeat zone, an expected pace under a predetermined condition, an expected pitch under a predetermined condition, an expected stride under a predetermined condition, an expected time under a predetermined condition (a lap time or a split time), expected calorie consumption under a predetermined condition, an automatically generated target, an ultraviolet quantity, . . . , and the like.

The processing section 120 of the above-described embodiment may display one or a plurality of performance items for each event.

### 3-7. Sensor Types

The electronic apparatus 1 of the above-described embodiment may use at least one of the following various sensors. In other words, the various sensors are, for example, an acceleration sensor, a GPS (GNSS) sensor, an angular velocity sensor, a speed sensor, a heartbeat sensor (a chest belt or the like), a pulse sensor (a sensor performing measurement at locations other than the heart), a pedometer, a pressure sensor, an altitude sensor, a temperature sensor (an atmospheric temperature sensor or a body temperature sensor), a geomagnetic sensor, a weight meter (which is used as an external device of the electronic apparatus 1), an ultraviolet sensor, a perspiration sensor, a blood pressure sensor, a blood oxygen concentration (SpO₂) sensor, a lactic acid sensor, and a blood sugar level sensor.

### 3-8. Data Collecting Method

The processing section 120 of the above-described embodiment may display at least one of an average value, a representative value (the maximum value or the minimum value), a total, and changes of data in a display object period as data (activity amount data or performance data) regarding at least one item.

For example, the processing section 120 of the above-described embodiment may display the worst data on the day, the best data on the day, and average data on the day as data on the day regarding a certain item, and may display the worst data for a week, the best data for a week, and average data for a week as data for a week regarding the same item.

For example, the processing section 120 of the above-described embodiment may display a split pace (an average pace for the entire running course), may display an average pace for a plurality of number of times of running, and may display an average pace (an average pace for overall running) for a month.

The processing section 120 of the above-described embodiment displays actually measured data up to the present time based on the history of sensing data or the sensing data as data regarding at least one item, but may display future expected data based on the history of the sensing data. The expected data is, for example, an expected number of steps under a predetermined condition, an expected movement distance under a predetermined condition, and a target value which is automatically generated.

The processing section 120 of the above-described embodiment may score data regarding at least one item and may display a score. At least one item may be evaluated according to a predetermined reference, and an evaluation result may be displayed.

### 3-9. Customizing

At least some of the display content (including a display object period, a display object item, a display aspect, a collecting method, a display order, and the like) in the processing section 120 of the above-described embodiment may be set in advance by a user (customizable).

### 4. Other Modification Examples

The invention is not limited to the above-described embodiment, and may be variously modified within the scope of the spirit of the invention.

For example, in the above-described embodiment, some of the functions of the server 4 may be installed in the information terminal 2 or the electronic apparatus 1, and some of the functions of the information terminal 2 or the electronic apparatus 1 may be installed in the server 4. In the above-described embodiment, some or all of the functions of the electronic apparatus 1 may be installed in the information terminal 2, and some or all of the functions of the information terminal 2 may be installed in the electronic apparatus 1.

Well-known functions of a smart phone, for example, a camera function, a call function, and a communication function may be installed in the electronic apparatus 1 or the information terminal 2.

Regarding an application (physical activity) of the electronic apparatus 1 or the information terminal 2, the electronic apparatus 1 or the information terminal 2 may be applied not only to walking or running, but also to climbing, skiing (including cross-country and ski jumping), bicycling, tennis, swimming, diet, rehabilitation, skating, golf, baseball, football, motorcycling, boating (rowboating), yachting, trail running, paragliding, dogsledding, horse riding, and the like. Different items may be logged depending on each application, and a user may select an item.

The electronic apparatus 1 or the information terminal 2 may be configured as portable information apparatuses of various types, such as a wrist type electronic apparatus, an earphone type electronic apparatus, a ring type electronic apparatus, a pendant type electronic apparatus, an electronic apparatus attached to a sport appliance and used, a smart phone, and a head mounted display (HMD).

The electronic apparatus 1 or the information terminal 2 may perform a notification of information for a user through image display, may perform a notification through sound output or by using vibration, light, or a color (light emission from an LED or a display color of a display), and may perform a notification through a combination of at least two of image display, sound output, vibration, light, and a color.

In the above-described embodiment, a global positioning system (GPS) is used, but a global navigation satellite system (GNSS) may be used. For example, one or two or more of satellite positioning systems such as a European geostationary-satellite navigation overlay service (EGNOS), a quasi zenith satellite system (QZSS), a global navigation satellite system (GLONASS), GALILEO, a BeiDou navigation satellite system (BeiDou) may be used. As at least one of the satellite positioning systems, a satellite-based augmentation system (SBAS) such as European geostationary-satellite navigation overlay service (EGNOS) or a wide area augmentation system (WAAS) may be used.

The above-described embodiment and modification examples are only examples, and the invention is not limited thereto. For example, the embodiment and the respective modification examples may be combined with each other as appropriate.

The invention includes substantially the same configuration (for example, a configuration in which functions, methods, and results are the same, or a configuration in which objects and effects are the same) as the configuration described in the embodiment. The invention includes a configuration in which an inessential part of the configuration described in the embodiment is replaced with another part. The invention includes a configuration which achieves the same operation and effect or a configuration capable of achieving the same object as in the configuration described in the embodiment. The invention includes a configuration in which a well-known technique is added to the configuration described in the embodiment.

What is claimed is:

1. A portable electronic apparatus comprising:
an input section that receives an input of an operation from a user;
a sensor that performs detection related to physical activity of the user;
a display section that displays data by using an output from the sensor; and
a processing section that maintains a display object item of data displayed on the display section to be a predetermined item and changes a display object period of the data displayed on the display section to a period (differing period) whose length is different from a length of a predetermined period in a case where the input section receives an input of a first operation when the data in the predetermined period regarding the predetermined item is displayed on the display section.

2. The portable electronic apparatus according to claim 1,
wherein the first operation is formed of a single action.

3. The portable electronic apparatus according to claim 2,
wherein the input section includes a button, and
wherein the first operation is an operation of pressing the button once.

4. The portable electronic apparatus according to claim 1,
wherein, in a case where the input section receives an input of a second operation when data in the differing period regarding the predetermined item is displayed on the display section, the processing section maintains the display object item to be the predetermined item, and changes the display object period from the differing period to the predetermined period.

5. The portable electronic apparatus according to claim 4,
wherein the second operation is formed of a single action.

6. The portable electronic apparatus according to claim 5,
wherein the input section includes a button, and
wherein the second operation is an operation of pressing the button once.

7. The portable electronic apparatus according to claim 4,
wherein the second operation is the same as the first operation.

8. The portable electronic apparatus according to claim 1,
wherein the predetermined period is a part of the differing period.

9. The portable electronic apparatus according to claim 8,
wherein the predetermined period is a day, and the differing period is a week.

10. The portable electronic apparatus according to claim 1,
wherein the display object item includes at least one of an item regarding an amount of activity in the life and an item regarding performance in motion.

11. The portable electronic apparatus according to claim 10,
wherein the item regarding an amount of activity includes at least one of a distance, a time, the number of steps, a pace, a pitch, a stride, the number of increasing stories, the number of increasing stairs, a heartbeat, oxygen intake, an amount of perspiration, water intake, calorie consumption, calorie intake, calories balance, a weight, a waist size, a sleeping time, sleep quality, balance between sleep quality, balance between an excitation time and a relaxing time, a target achievement level, and an ultraviolet quantity.

12. The portable electronic apparatus according to claim 10,
wherein the item regarding performance includes at least one of a distance, a time, the number of steps, a pace, a pitch, a stride, increasing altitude, decreasing altitude, an elevation, gradient, the number of times of training, a target achievement level, an attitude, left/right difference, foot contact time, a directly-below landing ratio, propulsion efficiency, slow turnover of the legs, a landing brake quantity, landing impact, a heart rate, oxygen intake, an amount of perspiration, water intake, calorie consumption, an expected motion distance under a predetermined condition, the time until reaching a predetermined heartbeat zone, an expected pace under a predetermined condition, an expected pitch under a predetermined condition, an expected stride under a predetermined condition, an expected time under a predetermined condition, expected calorie consumption under a predetermined condition, an automatically generated target, and an ultraviolet quantity.

13. The portable electronic apparatus according to claim 1,
wherein the portable electronic apparatus is attachable to a predetermined part of the user.

14. The portable electronic apparatus according to claim 13,
wherein the predetermined part is the arm or the wrist of the user.

15. A display method for a portable electronic apparatus, the method comprising:
a causing an input section to receive an input of an operation from a user;
causing a sensor to perform detection related to physical activity of the user;
causing a display section to display data by using an output from the sensor; and
maintaining a display object item of data displayed on the display section to be a predetermined item and changing a display object period of the data displayed on the display section to a period (differing period) whose length is different from a length of a predetermined period in a case where an input of a first operation is received when the data in the predetermined period regarding the predetermined item is displayed on the display section.

16. The display method for a portable electronic apparatus according to claim 15,
wherein the first operation is formed of a single action.

17. The display method for a portable electronic apparatus according to claim 16,
wherein the first operation is an operation of pressing a button once.

18. The display method for a portable electronic apparatus according to claim 15,
wherein, in the changing of the display object period, in a case where an input of a second operation is received when data in the differing period regarding the predetermined item is displayed on the display section, the display object item is maintained to be the predetermined item, and the display object period is changed from the differing period to the predetermined period.

19. The display method for a portable electronic apparatus according to claim 18,
wherein the second operation is formed of a single action.

20. The display method for a portable electronic apparatus according to claim 19,
wherein the second operation is an operation of pressing a button once.

21. The display method for a portable electronic apparatus according to claim 19,
wherein the second operation is the same as the first operation.

22. The display method for a portable electronic apparatus according to claim 15,
wherein the predetermined period is a part of the differing period.

23. The display method for a portable electronic apparatus according to claim 22,
wherein the predetermined period is a day, and the differing period is a week.

24. The display method for a portable electronic apparatus according to claim 15,
wherein the display object item includes at least one of an item regarding an amount of activity in the life and an item regarding performance in motion.
